(19) United States
(12) Patent Application Publication
(10) Pub. No.: US 2026/0278790 A1
(43) Pub. Date: Sep. 17, 2026

(54) SYSTEMS AND METHODS FOR GENERATING EXTENDED DEPTH OF FIELD MEDICAL VIDEO

(71) Applicant: Stryker Corporation, Portage, MI (US)

(72) Inventors: Marc ANDRÉ, Spiegel b. Bern (CH); Benjamin Hyman FEINGOLD, San Francisco, CA (US); Levey Trac TRAN, Denver, CO (US)

(73) Assignee: Stryker Corporation, Portage, MI (US)

(21) Appl. No.: 19/567,712

(22) Filed: Mar. 16, 2026

Related U.S. Application Data
(60) Provisional application No. 63/773,301, filed on Mar. 17, 2025.

(51) Int. Cl. G06T 7/00; G06T 5/20; G06T 5/50; G06T 5/70; G06T 7/20; G06T 7/50; G16H 30/40

(52) U.S. Cl. CPC G06T 7/0012; G06T 5/20; G06T 5/50; G06T 5/70; G06T 7/20; G06T 7/50; G16H 30/40; G06T 2207/10068; G06T 2207/20221

(57) ABSTRACT

A method for medical imaging includes, at a computing system: receiving a series of video frames of a region of tissue of a subject captured using different focal plane settings of a camera; generating focus maps for at least some of the video frames of the series of video frames, wherein the focus maps are generated using at least one smoothing filter; generating extended depth of field video frames by combining image data from video frames of the series of video frames based on the focus maps; and displaying the extended depth of field video frames.

### CROSS-REFERENCE TO RELATED APPLICATIONS

This application claims the benefit of U.S. Provisional Application No. 63/773,301, filed Mar. 17, 2025, the entire contents of which are incorporated herein by reference.

### FIELD

This disclosure relates generally to medical imaging and, more specifically, to medical image processing.

### BACKGROUND

Minimally invasive surgery generally involves the use of a high-definition camera coupled to an endoscope inserted through a small incision into a subject to provide a surgeon with a clear and precise view within the body with minimal tissue damage. The endoscope emits light from its distal end to illuminate the surgical cavity and receives light reflected or emitted by tissue within the surgical cavity through a lens or window located at the distal end of the endoscope. The confined nature of the surgical cavity often leads to in-focus and out-of-focus regions of a field of view of the camera. For example, a foreground of a field of view may be in-focus, but a background of the field of view may be out-of-focus. The camera may include the ability to adjust its depth of field to select which regions of the field of view are in-focus. For example, a camera may have an auto-focus function that automatically adjusts a focal plane setting of the camera such that as much of the field of view as possible is in-focus, but this may still result in some regions of the field of view being out-of-focus.

### SUMMARY

Systems and methods may include combining video frames captured using different focal plane settings into an extended depth of field frame in which more of the field of view is in-focus. A focal plane setting of a camera may be adjusted frame-to-frame such that different frames have different ranges of in-focus depths. Focus maps may be generated for each frame to characterize the degree of focus of different portions of each frame. The focus maps may be used to combine frames captured using different focal plane settings into an extended depth of field frame in which the in-focus portions of the different frames dominate, thereby resulting in more of the field of view being in-focus compared to each frame used to generate the extended depth of field frame.

According to an aspect, a method for medical imaging includes, at a computing system: receiving a series of video frames of a region of tissue of a subject captured using different focal plane settings of a camera; generating focus maps for at least some of the video frames of the series of video frames, wherein the focus maps are generated using at least one smoothing filter; generating extended depth of field video frames by combining image data from video frames of the series of video frames based on the focus maps; and displaying the extended depth of field video frames.

For a given extended depth of field video frame generated from a given set of the video frames, a pixel value of the given extended depth of field video frame may be a function of corresponding pixel values of the given set of the video frames and corresponding values of focus maps generated for the given set of the video frames.

The at least one smoothing filter may include at least one spatial filter. The at least one smoothing filter may include at least one temporal filter. At least one of the focus maps may be generated from a luminance channel of a respective video frame. At least one of the focus maps may be generated based on fewer than all color channels of the respective video frame.

The extended depth of field video frames may be generated in an extended depth of field mode, and the method may include determining an excessive motion of the camera, and in response to determining the excessive motion of the camera, exiting the extended depth of field mode. The method may include determining an excessive motion of the camera, and in response to determining the excessive motion of the camera, reducing a number of video frames used to generate each extended depth of field video frame.

Generating the extended depth of field video frames may include generating weights based on the filtered focus maps and combining image data from the video frames based on the weights. Each focus map may be generated based on a luminance channel of a respective video frame, and the extended depth of field video frames may be generated using respective weights to combine image data from the luminance channel of the respective video frame. The series of video frames may include first imaging modality video frames and second imaging modality frames, and the extended depth of field video frames may be generated based only on the first imaging modality video frames.

The method may include adjusting the focal plane setting of the camera based on at least one of the focus maps. The method may include determining at least one depth measurement based on the focal plane setting of the camera. The method may include adjusting the focal plane setting of the camera based on an output of at least one distance sensor.

Each focus map may include an array of values corresponding to estimates of contrast in a plurality of locations of a respective video frame. The method may include generating estimates of tissue depth based on the focus maps. Generating estimates of tissue depth based on the focus maps may include determining estimates of tissue depth for different imaging modalities and comparing the estimates of tissue depth for the different imaging modalities to determine a depth of a feature of interest below a surface of tissue.

The camera may include a liquid lens for providing the different focal plane settings. The method may include controlling the liquid lens based on a camera temperature. The camera may be an endoscopic camera or an open field camera. The method may include segmenting at least one frame and generating the extended depth of field video frames by combining image data from video frames of the series of video frames based on the segmentation of the at least one frame.

According to an aspect, a system for medical imaging includes one or more processors, at least one display, and memory storing one or more programs that include instructions for execution by the one or more processors for causing the system to: receive a series of video frames of a region of tissue of a subject captured using different focal plane settings of a camera; generate focus maps for at least some of the video frames of the series of video frames wherein the focus maps are generated using at least one smoothing filter; generate extended depth of field video frames by combining image data from video frames of the series of video frames based on the focus maps; and display the extended depth of field video frames on the at least one display.

For a given extended depth of field video frame generated from a given set of the video frames, a pixel value of the given extended depth of field video frame may be a function of corresponding pixel values of the given set of the video frames and corresponding values of focus maps generated for the given set of the video frames.

The at least one smoothing filter may include at least one spatial filter. The at least one smoothing filter may include at least one temporal filter. At least one of the focus maps may be generated from a luminance channel of a respective video frame. At least one of the focus maps may be generated based on fewer than all color channels of the respective video frame.

The extended depth of field video frames may be generated in an extended depth of field mode, and the one or more programs may include instructions for determining an excessive motion of the camera, and in response to determining the excessive motion of the camera, exiting the extended depth of field mode. The one or more programs may include instructions for determining an excessive motion of the camera, and in response to determining the excessive motion of the camera, reducing a number of video frames used to generate each extended depth of field video frame.

Generating the extended depth of field video frames may include generating weights based on the filtered focus maps and combining image data from the video frames based on the weights. Each focus map may be generated based on a luminance channel of a respective video frame, and the extended depth of field video frames may be generated using respective weights to combine image data from the luminance channel of the respective video frame. The series of video frames may include first imaging modality video frames and second imaging modality frames, and the extended depth of field video frames may be generated based only on the first imaging modality video frames.

The one or more programs may include instructions for adjusting the focal plane setting of the camera based on at least one of the focus maps. The one or more programs may include instructions for determining at least one depth measurement based on the focal plane setting of the camera. The one or more programs may include instructions for adjusting the focal plane setting of the camera based on an output of at least one distance sensor.

Each focus map may include an array of values corresponding to estimates of contrast in a plurality of locations of a respective video frame. The one or more programs may include instructions for generating estimates of tissue depth based on the focus maps. Generating estimates of tissue depth based on the focus maps may include determining estimates of tissue depth for different imaging modalities and comparing the estimates of tissue depth for the different imaging modalities to determine a depth of a feature of interest below a surface of tissue.

The camera may include a liquid lens for providing the different focal plane settings. The one or more programs may include instructions for controlling the liquid lens based on a camera temperature. The camera may be an endoscopic camera or an open field camera. The one or more programs may include instructions for segmenting at least one frame and generating the extended depth of field video frames by combining image data from video frames of the series of video frames based on the segmentation of the at least one frame.

According to an aspect, a method for medical imaging includes: receiving a series of video frames of a region of tissue of a subject captured using different focal plane settings of a camera; generating focus maps for at least some of the video frames of the series of video frames based on only luminance channels of the video frames or only one color channel of the video frames; generating extended depth of field video frames by combining image data from video frames of the series of video frames based on the focus maps; and displaying the extended depth of field video frames.

For a given extended depth of field video frame generated from a given set of the video frames, a pixel value of the given extended depth of field video frame may be a function of corresponding pixel values of the given set of the video frames and corresponding values of focus maps generated for the given set of the video frames. Generating the extended depth of field video frames may include spatially filtering at least one focus map. Generating the extended depth of field video frames may include temporally filtering at least one focus map.

The extended depth of field video frames may be generated in an extended depth of field mode, and the method may include determining an excessive motion of the camera, and in response to determining the excessive motion of the camera, exiting the extended depth of field mode. The method may include determining an excessive motion of the camera, and in response to determining the excessive motion of the camera, reducing a number of video frames used to generate each extended depth of field video frame.

Generating the extended depth of field video frames may include generating weights based on the focus maps and combining image data from the video frames based on the weights. The series of video frames may include first imaging modality video frames and second imaging modality frames, and the extended depth of field video frames may be generated based only on the first imaging modality video frames. The method may include adjusting the focal plane setting of the camera based on at least one of the focus maps.

The method may include determining at least one depth measurement based on the focal plane setting of the camera. The method may include adjusting the focal plane setting of the camera based on an output of at least one distance sensor. Each focus map may include an array of values corresponding to estimates of contrast in a plurality of locations of a respective video frame. The method may include generating estimates of tissue depth based on the focus maps. Generating estimates of tissue depth based on the focus maps may include determining estimates of tissue depth for different imaging modalities and comparing the estimates of tissue depth for the different imaging modalities to determine a depth of a feature of interest below a surface of tissue.

The camera may include a liquid lens for providing the different focal plane settings. The method may include controlling the liquid lens based on a camera temperature. The camera may be an endoscopic camera. The camera may be an open field camera.

The method may include segmenting at least one frame and generating the extended depth of field video frames by combining image data from video frames of the series of video frames based on the segmentation of the at least one frame.

According to an aspect, a system for medical imaging includes one or more processors, at least one display, and memory storing one or more programs that include instructions for execution by the one or more processors for causing the system to: receive a series of video frames of a region of tissue of a subject captured using different focal plane settings of a camera; generate focus maps for at least some of the video frames of the series of video frames based on only luminance channels of the video frames or only one color channel of the video frames; generate extended depth of field video frames by combining image data from video frames of the series of video frames based on the focus maps; and display the extended depth of field video frames on the at least one display.

For a given extended depth of field video frame generated from a given set of the video frames, a pixel value of the given extended depth of field video frame may be a function of corresponding pixel values of the given set of the video frames and corresponding values of focus maps generated for the given set of the video frames. Generating the extended depth of field video frames may include spatially filtering at least one focus map. Generating the extended depth of field video frames may include temporally filtering at least one focus map.

The extended depth of field video frames may be generated in an extended depth of field mode, and the one or more programs may include instructions for determining an excessive motion of the camera, and in response to determining the excessive motion of the camera, exiting the extended depth of field mode. The one or more programs may include instructions for determining an excessive motion of the camera, and in response to determining the excessive motion of the camera, reducing a number of video frames used to generate each extended depth of field video frame.

Generating the extended depth of field video frames may include generating weights based on the focus maps and combining image data from the video frames based on the weights. The series of video frames may include first imaging modality video frames and second imaging modality frames, and the extended depth of field video frames may be generated based only on the first imaging modality video frames. The one or more programs may include instructions for adjusting the focal plane setting of the camera based on at least one of the focus maps.

The one or more programs may include instructions for determining at least one depth measurement based on the focal plane setting of the camera. The one or more programs may include instructions for adjusting the focal plane setting of the camera based on an output of at least one distance sensor. Each focus map may include an array of values corresponding to estimates of contrast in a plurality of locations of a respective video frame. The one or more programs may include instructions for generating estimates of tissue depth based on the focus maps. Generating estimates of tissue depth based on the focus maps may include determining estimates of tissue depth for different imaging modalities and comparing the estimates of tissue depth for the different imaging modalities to determine a depth of a feature of interest below a surface of tissue.

The camera may include a liquid lens for providing the different focal plane settings. The method may include controlling the liquid lens based on a camera temperature. The camera may be an endoscopic camera. The camera may be an open field camera.

The one or more programs may include instructions for segmenting at least one frame and generating the extended depth of field video frames by combining image data from video frames of the series of video frames based on the segmentation of the at least one frame.

According to an aspect, a method for medical imaging includes receiving a series of video frames comprising: first imaging modality video frames captured by a camera at one or more first imaging modality focal plane settings, and second imaging modality video frames captured by the camera at one or more second imaging modality focal plane settings that are different from the one or more first imaging modality focal plane settings; combining image data from at least some of the first imaging modality video frames with image data from at least some of the second imaging modality video frames to generate combined imaging modality video frames; and displaying the combined imaging modality video frames.

The method may include generating at least one focus estimate for at least one of the first imaging modality video frames and setting at least one of the one or more first imaging modality focal plane settings based on the at least one focus estimate. The method may include generating at least one focus estimate for at least one of the second imaging modality video frames and setting at least one of the one or more second imaging modality focal plane settings based on the at least one focus estimate for the at least one of the second imaging modality video frames. The one or more first imaging modality focal plane settings may include a plurality of different first imaging modality focal plane settings, and the method may include: generating focus maps for at least some of the first imaging modality video frames; and generating extended depth of field first imaging modality video frames by combining image data from first imaging modality video frames based on the focus maps.

According to an aspect, a system for medical imaging includes one or more processors, at least one display, and memory storing one or more programs that include instructions for execution by the one or more processors for causing the system to: receive a series of video frames comprising: first imaging modality video frames captured by a camera at one or more first imaging modality focal plane settings, and second imaging modality video frames captured by the camera at one or more second imaging modality focal plane settings that are different from the one or more first imaging modality focal plane settings; combine image data from at least some of the first imaging modality video frames with image data from at least some of the second imaging modality video frames to generate combined imaging modality video frames; and display the combined imaging modality video frames on the at least one display.

The one or more programs may include instructions for generating at least one focus estimate for at least one of the first imaging modality video frames and setting at least one of the one or more first imaging modality focal plane settings based on the at least one focus estimate. The one or more programs may include instructions for generating at least one focus estimate for at least one of the second imaging modality video frames and setting at least one of the one or more second imaging modality focal plane settings based on the at least one focus estimate for the at least one of the second imaging modality video frames. The one or more first imaging modality focal plane settings may include a plurality of different first imaging modality focal plane settings, and the one or more programs may include instructions for: generating focus maps for at least some of the first imaging modality video frames; and generating extended depth of field first imaging modality video frames by combining image data from first imaging modality video frames based on the focus maps.

According to an aspect, a method for medical imaging includes, at a computing system: receiving a series of video frames of a region of tissue of a subject captured using different focal plane settings of a camera; converting at least some of the video frames of the series of video frames into detail and base layers; generating extended depth of field video frames by combining detail layers of a plurality of the video frames and combining base layers of the plurality of video frames; and displaying the extended depth of field video frames.

The camera may include a liquid lens for providing the different focal plane settings. The method may include controlling the liquid lens based on a camera temperature. The camera may be an endoscopic camera. The camera may be an open field camera.

According to an aspect, a system for medical imaging includes one or more processors, at least one display, and memory storing one or more programs that include instructions for execution by the one or more processors for causing the system to: receive a series of video frames of a region of tissue of a subject captured using different focal plane settings of a camera; convert at least some of the video frames of the series of video frames into detail and base layers; generate extended depth of field video frames by combining detail layers of a plurality of the video frames and combining base layers of the plurality of video frames; and display the combined imaging modality video frames on the at least one display.

The camera may include a liquid lens for providing the different focal plane settings. The one or more programs may include instructions for controlling the liquid lens based on a camera temperature. The camera may be an endoscopic camera. The camera may be an open field camera.

It will be appreciated that any of the variations, aspects, features, and options described in view of the systems apply equally to the methods and vice versa. It will also be clear that any one or more of the above variations, aspects, features, and options can be combined.

### BRIEF DESCRIPTION OF THE FIGURES

The invention will now be described, by way of example only, with reference to the accompanying drawings, in which:

FIG. 1 illustrates an exemplary imaging system for imaging tissue of a subject;

FIG. 2 illustrates an exemplary method for generating extended depth of field video frames by combining video frames captured with different focal plane settings;

FIG. 3 illustrates an exemplary imaging system that includes a camera having an adjustable lens assembly configured for adjusting a focal plane setting;

FIGS. 4A and 4B illustrate exemplary focus maps;

FIGS. 5A and 5B are schematic illustrations of the combining of endoscopic video frames of a target region using different focal plane settings to obtain an extended depth of field video frame;

FIG. 6 is a functional block diagram of an exemplary image processing unit illustrating an example of how frames may be combined to generate an extended depth of field frames;

FIG. 7 illustrates an example of the combination of two endoscopic imaging frames captured with different focal plane settings into an extended depth of field frame;

FIGS. 8A-8C illustrate examples of generating extended depth of field frames based on image decomposition;

FIG. 9 illustrates an exemplary method for automatically entering and exiting an extended depth of field mode based on the amount of motion in a series of video frames;

FIG. 10 illustrates an exemplary method for adjusting the number of frames used to generate each extended depth of field frame;

FIG. 11 illustrates an exemplary method for multi-modality imaging using different focal plane settings for different modalities;

FIG. 12 illustrates an exemplary method for generating estimates of target depth using focus maps;

FIGS. 13A and 13B illustrate the application of the method of FIG. 12 to a series of three video frames; and

FIG. 14 is a functional block diagram of an exemplary computing system.

### DETAILED DESCRIPTION

In the following description of the various examples, reference is made to the accompanying drawings in which are shown, by way of illustration, specific examples that can be practiced. The description is presented to enable one of ordinary skill in the art to make and use the invention and is provided in the context of a patent application and its requirements. Various modifications to the described examples will be readily apparent to those persons skilled in the art, and the generic principles herein may be applied to other examples. Thus, the present invention is not intended to be limited to the examples shown but is to be accorded the widest scope consistent with the principles and features described herein.

According to various aspects, systems and methods disclosed herein include combining medical images (as used herein, the term “image” encompasses single snapshot images and video frames) captured using different focal plan settings into extended depth of field frames. A focal plane setting of a camera may be adjusted for capturing different images of a region of interest of a subject such that the different images have different ranges of depth that are in-focus. The different ranges of depths that are in focus may result in different portions of the region of interest being in-focus in different images. Focus maps may be generated for the different images to characterize the degree of focus across the images. The focus maps may be used to combine the images into an extended depth of field image that has a greater proportion of the region of interest of the subject in-focus.

The focal plane setting may be adjusted using an adjustable lens assembly of the camera. The images may be video frames that may be combined to generate extended depth of field video frames. As such, the adjustable lens assembly may be configured to enable focal plane setting adjustment at a rate sufficient for generating extended depth of field video frames. For example, the adjustable lens assembly may include a liquid lens that can be driven to adjust focal plane settings at high rates, including video frame rates. The adjustable lens assembly may include a motorized lens assembly configured for high-speed focal plane setting adjustment that is sufficient for generating extended depth of field video frames. The motorized lens assembly may include, for example, an electric motor, a magnetic motor, or an ultrasonic motor.

By combining images captured using different focal plane settings into an extended depth of field frame, more of a field of view may be in-focus. Additionally, or alternatively, the ability to combine different ranges of in-focus depths into an extended depth of field may enable the use of a larger aperture and/or optics that provide more light to the imaging sensor(s), which may increase image quality.

In the following description, it is to be understood that the singular forms “a,” “an,” and “the” used in the following description are intended to include the plural forms as well, unless the context clearly indicates otherwise. It is also to be understood that the term “and/or” as used herein refers to and encompasses any and all possible combinations of one or more of the associated listed items. It is further to be understood that the terms “includes,” “including,” “comprises,” and/or “comprising,” when used herein, specify the presence of stated features, integers, steps, operations, elements, components, and/or units but do not preclude the presence or addition of one or more other features, integers, steps, operations, elements, components, units, and/or groups thereof.

Certain aspects of the present disclosure include process steps and instructions described herein in the form of an algorithm. It should be noted that the process steps and instructions of the present disclosure could be embodied in software, firmware, or hardware and, when embodied in software, could be downloaded to reside on and be operated from different platforms used by a variety of operating systems. Unless specifically stated otherwise as apparent from the following discussion, it is appreciated that, throughout the description, discussions utilizing terms such as “processing,” “computing,” “calculating,” “determining,” “displaying,” “generating,” or the like refer to the action and processes of a computer system, or similar electronic computing device, that manipulates and transforms data represented as physical (electronic) quantities within the computer system memories or registers or other such information storage, transmission, or display devices.

The present disclosure in some aspects also relates to devices or systems for performing the operations herein. The devices or systems may be specially constructed for the required purposes, may comprise a general-purpose computer selectively activated or reconfigured by a computer program stored in the computer, or may include any combination thereof. Computer instructions for performing the operations herein can be stored in any combination of non-transitory, computer-readable storage medium, such as, but not limited to, any type of disk, including USB flash drives, external hard drives, optical disks, CD-ROMs, magnetic-optical disks, read-only memories (ROMs), random access memories (RAMs), EPROMs, EEPROMs, magnetic or optical cards, or any type of media suitable for storing electronic instructions, and each coupled to a computer system bus. One or more instructions for performing the operations herein may be implemented in or executed by one or more Application Specific Integrated Circuits (ASICs), Field Programmable Gate Arrays (FPGAs), Digital Signal Processing units (DSPs), Graphics Processing Units (GPUs), Central Processing Units (CPUs), or any other suitable processing unit. Furthermore, the computers referred to herein may include a single processor or may be architectures employing multiple processor designs for increased computing capability.

The methods, devices, and systems described herein are not inherently related to any particular computer or other apparatus. Various general-purpose systems may also be used with programs in accordance with the teachings herein, or it may prove convenient to construct a more specialized apparatus to perform the required method steps. The required structure for a variety of these systems will appear from the description below. In addition, the present invention is not described with reference to any particular programming language. It will be appreciated that a variety of programming languages may be used to implement the teachings of the present invention as described herein.

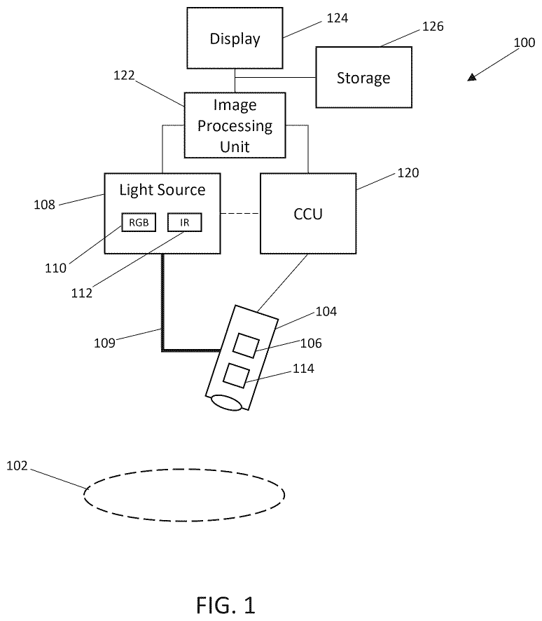

FIG. 1 illustrates an exemplary imaging system 100 for imaging a target region 102 of tissue of a subject during an imaging session, which can be an imaging session occurring during a surgical procedure or a non-surgical medical procedure. The system 100 includes a medical camera 104 (also referred to herein as a camera). The camera 104 has at least one image sensor 106 configured to capture images (e.g., single snapshot images and/or a series of video frames) of a target region. (The terms image and frame are used interchangeably throughout.) The camera 104 may include at least one adjustable lens 114 for adjusting a focal plane setting of the camera 104. The at least one adjustable lens 114 may include an electronically controlled adjustable lens 114 that may be controlled electronically to adjust the focal plane setting of the camera 104. The at least one adjustable lens 114 may be manually adjustable (in addition to, or alternatively to, being electronically adjustable) or may include at least one manually adjustable lens. The camera 104 can be a hand-held device, such as a hand-held open-field camera or a hand-held endoscopic camera, or can be a device mounted or attached to a mechanical support arm.

The camera 104 may be connected to a camera control unit (CCU) 120, which may generate one or more single snapshot images and/or video frames (referred to herein collectively as images) from imaging data generated by the camera 104. The CCU 120 may send control signals to the camera 104, such as to control the adjustable lens 114 to achieve different focal plane settings, to control a mechanical shutter of the camera 104, and/or to adjust a gain of the at least one image sensor 106. In some variations, the functionalities of the CCU 120 and the camera 104 are incorporated into a single device.

The images generated by the camera control unit 120 may be transmitted to an image processing unit 122 that may apply one or more image processing techniques described further below to the images generated by the camera control unit 120. Optionally, the functionalities of the camera control unit 120 and the image processing unit 122 described herein are integrated into a single device. The image processing unit 122 (and/or the camera control unit 120) may be connected to one or more displays 124 for displaying the one or more images generated by the camera control unit 120 or one or more images or other visualizations generated based on the images generated by the camera control unit 120. The image processing unit 122 (and/or the camera control unit 120) may store the one or more images generated by the camera control unit 120 or one or more images or other visualizations generated based on the images generated by the camera control unit 120 in one or more storage devices 126. The one or more storage devices can include one or more local memories, one or more remote memories, a recorder or other data storage device, a printer, and/or a picture archiving and communication system (PACS). The system 100 may additionally or alternatively include any suitable systems for communicating and/or storing images and image-related data.

The images generated using camera control unit 120 may be described herein as visible (e.g., white) light images and/or fluorescence images. However, it is to be understood that the imaging system 100 of FIG. 1 may be configured to generate images and/or video frames according to any other suitable imaging modality, including but not limited to radiation imaging (e.g., X-ray images gathered from imaging procedures such as CT scans, PET scans, etc.), MRI, and/or ultrasound imaging. Thus, the image processing unit 122 may be configured to process medical images of various modalities, such as visible light images, fluorescence images, X-ray images, MRI images, ultrasound images, etc.

The imaging system 100 may include a light source 108 configured to generate light that is directed to the field of view to illuminate the target region 102. Light generated by the light source 108 can be provided to the camera 104 by a light cable 109. The camera 104 may include one or more optical components, such as one or more lenses, fiber optics, light pipes, etc., for directing the light received from the light source 108 to the tissue. The camera 104 may be an endoscopic camera that includes an endoscope that includes one or more optical components for conveying the light to a scene within a surgical cavity into which the endoscope is inserted. The camera 104 may be an open-field camera and may include one or more lenses that direct the light toward the field of view of the open-field imager.

The light source 108 includes one or more visible light emitters 110 that emit visible light in one or more visible wavebands (e.g., full spectrum visible light, narrow band visible light, or other portions of the visible light spectrum). The visible light emitters 110 may include one or more solid state emitters, such as LEDs and/or laser diodes. The visible light emitters 110 may include red, green, and blue (or other color component) LEDs or laser diodes that in combination generate white light or other illumination needed for reflected light imaging. These color component light emitters may be centered around the same wavelengths around which the camera 104 is centered. For example, in variations in which the camera 104 includes a single chip, single color image sensor having an RGB color filter array deposited on its pixels, the red, green, and blue light sources may be centered around the same wavelengths around which the RGB color filter array is centered. As another example, in variations in which the camera 104 includes a three-chip, three-sensor (RGB) color camera system, the red, green, and blue light sources may be centered around the same wavelengths around which the red, green, and blue image sensors are centered.

The light source 108 can include one or more excitation light emitters 112 configured to emit excitation light suitable for exciting intrinsic fluorophores and/or extrinsic fluorophores (e.g., a fluorescence imaging agent that has been introduced into the subject) located in the tissue being imaged. The excitation light emitters 112 may include, for example, one or more LEDs, laser diodes, arc lamps, and/or illuminating technologies of sufficient intensity and appropriate wavelength to excite the fluorophores located in the object being imaged. For example, the excitation light emitter(s) may be configured to emit light in the near-infrared (NIR) waveband (such as, for example, approximately 805 nm light), though other excitation light wavelengths may be appropriate depending on the application.

The light source 108 may further include one or more optical elements that shape and/or guide the light output from the visible light emitters 110 and/or excitation light emitters 112. The optical components may include one or more lenses, mirrors (e.g., dichroic mirrors), light guides and/or diffractive elements, e.g., to help ensure a flat field over substantially the entire field of view of the camera 104.

The camera 104 may acquire reflected light images from visible light reflected from the tissue that is incident on the at least one image sensor 106 and/or fluorescence images from fluorescence light emitted by fluorophores in the tissue (which are excited by the fluorescence excitation light) that is incident on the at least one image sensor 106. The at least one image sensor 106 may include at least one solid state image sensor. The at least one image sensor 106 may include, for example, a charge coupled device (CCD), a CMOS sensor, a CID, or other suitable sensor technology. The at least one image sensor 106 may include a single image sensor (e.g., a grayscale image sensor or a color image sensor having an RGB color filter array deposited on its pixels). The at least one image sensor 106 may include multiple sensors, such as one sensor for detecting red light, one for detecting green light, and one for detecting blue light.

The camera control unit 120 can control timing of image acquisition by the camera 104. The camera 104 may be used to acquire both reflected light images and fluorescence images and the camera control unit 120 may control a timing scheme for the camera 104. The camera control unit 120 may be connected to the light source 108 for providing timing commands to the light source 108. Alternatively, the image processing unit 122 may control a timing scheme of the camera 104, the light source 108, or both.

The timing scheme of the camera 104 and the light source 108 may enable capture of reflected light images and fluorescence light images in an alternating fashion. In particular, the timing scheme may involve illuminating the tissue with illumination light and/or excitation light according to a pulsing scheme and processing the reflected light image and fluorescence image with a processing scheme that is synchronized and matched to the pulsing scheme to enable separation of the two types of images in a time-division multiplexed manner. Examples of such pulsing and image processing schemes have been described in U.S. Pat. No. 9,173,554, filed on Mar. 18, 2009, and titled “IMAGING SYSTEM FOR COMBINED FULL-COLOR REFLECTANCE AND NEAR-INFRARED IMAGING,” the contents of which are incorporated by reference in their entirety. However, other suitable pulsing and image processing schemes may be used to acquire reflected light video frames and fluorescence video frames. A reflected light video frame and a fluorescence video frame may be combined into a multi-modality video frame, such as an overlay of the fluorescence information from the fluorescence video frame on the visible light frame.

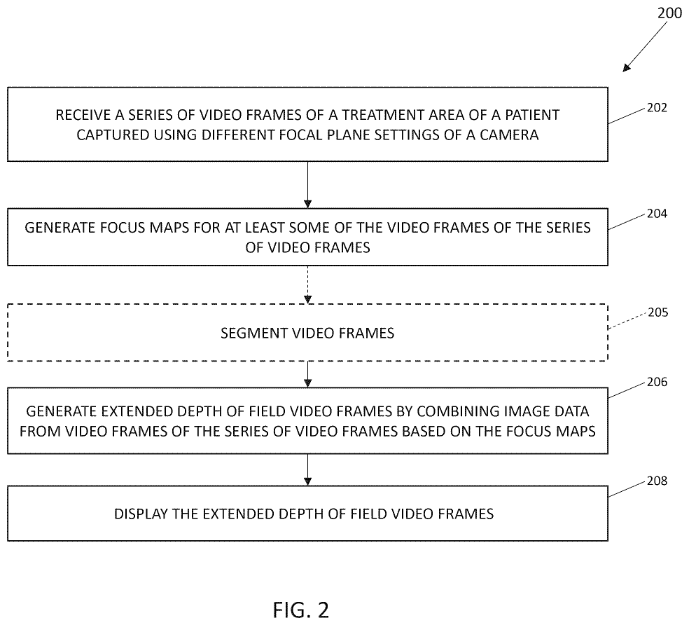

Imaging system 100 may be configured to generate extended depth of field video frames by combining video frames captured with different focal plane settings. FIG. 2 illustrates a method 200 that can be performed by imaging system 100 for generating extended depth of field video frames by combining video frames captured with different focal plane settings. At step 202, a series of video frames of a region of tissue of a subject captured using different focal plane settings of a camera are received. For example, with reference to FIG. 1, a series of video frames captured using camera 104 may be received by image processing unit 122 from camera control unit 120. At least one of the video frames in the series of video frames is captured using a different focal plane setting of a camera than at least one other video frame in the series of video frames. The camera may include an adjustable lens system that enables a focal plane setting to be adjusted such that video frames can be captured with different focal plane settings.

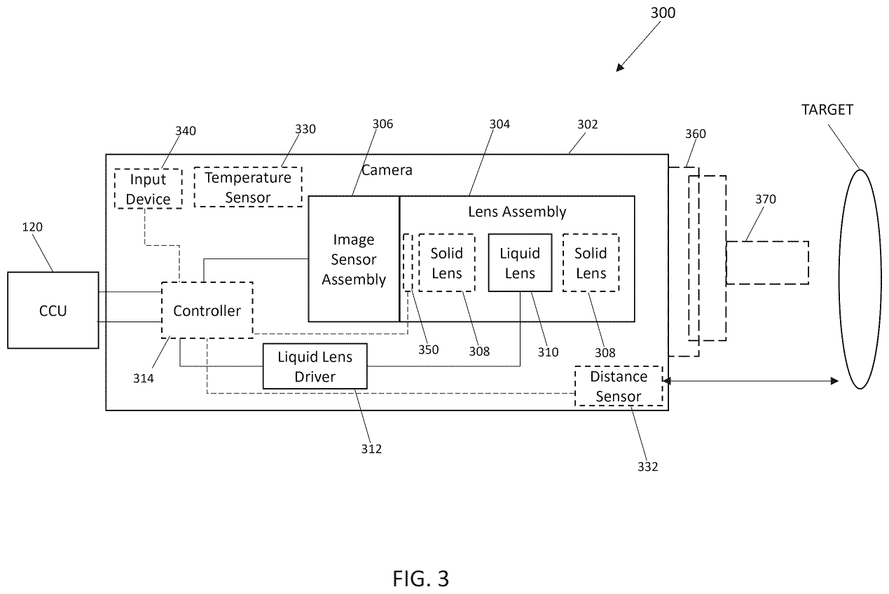

FIG. 3 illustrates an exemplary imaging system 300 that includes a camera 302 having an adjustable lens assembly 304 configured for adjusting a focal plane setting. Camera 302 may be used for camera 104 of system 100. Camera 302 may be an endoscopic imaging camera (which may include a coupler 360 for coupling to an endoscope 370), an open-field imaging camera, or any other type of medical imaging camera. Adjustable lens assembly 304 is positioned in front of an image sensor assembly 306 for focusing light onto the image sensor assembly 306 and may include at least one solid lens 308 (e.g., at least one glass lens) and at least one liquid lens 310. Power applied to the liquid lens 310 can be controlled (e.g., via voltage control or current control) to control a radius of curvature of the liquid lens, thereby controlling a focal plane setting of adjustable lens assembly 304. The one or more solid lenses 308 and the liquid lens 310 can be arranged in any suitable manner. For example, one or more solid lenses 308 may be positioned proximally of the liquid lens 310 (i.e., between the liquid lens 310 and the image sensor assembly 306) and/or one or more solid lenses 308 may be positioned distally of the liquid lens 310. Optionally, the liquid lens 310 may be spaced a sufficient distance away from the image sensor assembly 506 to mitigate heat from the assembly from heating the liquid lens enough to substantially affect focus performance. Adjustable lens assembly 304 may include an adjustable aperture 350.

The camera may include a liquid lens driver 312 that provides a controlled power to the liquid lens 310. The liquid lens driver 312 may receive control commands from CCU 120 (directly or via a controller 314 of the camera 302) that control the focal plane setting of the liquid lens 310. Alternatively, the controller 314 can control the focal plane setting of the liquid lens 310 itself, without receiving commands from the CCU 120. The power provided to the liquid lens 310 may be adjusted for purposes other than changing the focal plane setting of the liquid lens 310. For example, the power provided to the liquid lens 310 may be adjusted to account for changes in the temperature of the camera 302 determined, for example, based on reading from a temperature sensor 330. Optionally, the controller 314 may control the adjustable aperture 350.

The focal plane setting of the liquid lens 310 may be changed according to any suitable control scheme. The focal plane setting may be changed for each frame of a series of video frames or may be changed at a lower rate such that multiple frames are captured with a given focal plane setting. For example, where the video frames are generated at a frame rate, the focal plane setting may be changed at a rate of half the frame rate, a third of the frame rate, a quarter of the frame rate, a tenth of the frame rate, or any other fraction of the frame rate. The focal plane setting may be changed in predefined increments. For example, the focal plane setting may be increased by a predefined increment at least once and, optionally, multiple times up to a maximum focal plane setting of the liquid lens 310 and/or may be decreased by a predefined increment at least once and, optionally, multiple times down to a minimum focal plane setting of the liquid lens 310. The focal plane setting may be adjusted according to a repeating pattern. For example, the focal plane setting may be increased in increments from an initial focal plane setting to a desired maximum focal plane setting and then decreased in the same increments from the desired maximum focal plane setting back to the initial focal plane setting, and this process may be repeated for any desired duration (e.g., throughout an imaging session or while in an extended depth of field mode). A change from one focal plane setting to another may occur when the image sensor(s) is not collecting light from the scene, such as when a shutter of the camera is blocking light from the scene or when no illumination light is being provided to the scene.

Adjustable lens assembly 304 may include a motorized lens assembly configured for high-speed focal plane setting adjustment that is sufficient for generating extended depth of field video frames. The motorized lens assembly may include, for example, an electric motor, a magnetic motor, or an ultrasonic motor.

Optionally, focal plane setting adjustments may be dynamic. For example, the CCU 120 or the image processing unit 122 may analyze a given frame received from the camera 302 to determine an adjustment to the focal plane setting for capturing a subsequent frame. A given frame may be analyzed to determine a focus metric that estimates the degree of focus of the image data and may adjust the focal plane setting accordingly. The focus metric may be determined by analyzing high frequency components of a given frame, analyzing contrast within the frame, and/or using any other suitable focus estimate technique.

A dynamic focal plane setting process may be advantageous for accommodating different optical configurations—such as for accommodating different endoscopes used with a given endoscopic camera head. Different optical configurations may result in different ranges of in-focus depths for a given focal plane setting, and a dynamic focal plane setting process may tune the difference in focal plane setting from one frame to the next so as to provide sufficiently different ranges of in-focus depths from frame to frame. In other words, relatively small changes in the focal plane setting may be suitable for one optical configuration (e.g., a first type of endoscope) for providing ranges of in-focus depths that do not overlap too much but, yet, are not too distant from one another, whereas relatively large changes in the focal plane setting may be suitable for another optical configuration (e.g., a second type of endoscope) for providing ranges of in-focus depths that do not overlap too much but, yet, are not too distant from one another. A dynamic focal plane setting process may dynamically find the focal plane settings that are suitable for a given optical configuration.

Similarly, a dynamic focal plane setting adjustment process may be advantageous in accommodating different working distances. For example, a range of focal plane settings suitable at one working distance may not be suitable at another working distance. As such, by adjusting focal plane settings according to the degree of focus of frames in a dynamic fashion, the focal plane settings may be automatically tuned to the working distance.

Optionally, the focal plane setting may be controlled based on at least one distance measurement associated with a distance between the camera 302 and the target region of the subject. The at least one distance measurement may be generated by at least one distance sensor 332 of the camera 302. Optionally, multiple distance sensors 332 may be used to obtain distance measurements for multiple different locations of the target region. The multiple distance measurements may be used to set a range of focal plane settings used for capturing a series of video frames such that the focal plane settings used for generating the series of video frames capturing the target are tailored to the locations of the target region.

The different focal plane settings may be focal plane settings used during an automatic focusing process. The CCU 120 or image processing unit 122 may be configured to automatically focus the camera 104 by adjusting the focal plane setting of the camera 104 (e.g., by controlling the power applied to liquid lens 310) and analyzing focus maps for the resulting frames until a focus map meets predetermined criteria. Frames captured during the automatic focusing process may be combined according to method 200. Additionally, or alternatively, an automatic focusing process may be used until a suitable focus level is achieved and, thereafter, the focal plane settings may be adjusted according to predetermined adjustments tailored to generating extended depth of field frames according to method 200.

An automatic focusing process may include the CCU 120 resetting a focus characteristic threshold to a predetermined value and setting an initial focal plane setting by controlling the liquid lens driver 312 to set an initial power (via an initial set voltage or current). The CCU 120 may determine a focus characteristic value for a frame captured with the liquid lens 310 at the initial power (the focus characteristic value may be determined, for example, from a focus map generated for the frame, such as an average focus metric value for the focus map). The CCU 120 may determine whether the focus characteristic value of the frame is more than the focus characteristic threshold. If the focus characteristic value of the frame is higher than the focus characteristic threshold, the CCU 120 may set the focus characteristic threshold to the focus characteristic value of the frame. The CCU 120 may then control the liquid lens driver 312 to change the power being delivered to the liquid lens 310. The change in power (in what direction and in what quantity) may be determined based on how many times the loop of the algorithm has been traversed for that particular focusing process. At the start of the process, the power change may be in one direction (e.g., a decrease, which may cause the focal plane setting to increase). As the process proceeds through multiple loops, the power may be changed based on the focus characteristic history. Once the CCU 120 determines that a peak value for the focus characteristic has been passed, the CCU 120 will change direction and magnitude of the power adjustments for the liquid lens 310 to arrive back at the peak value of the focus characteristic. If the focus characteristic value of a given frame is not higher than the set focus characteristic threshold, the CCU 120 may control the liquid lens driver 312 to provide a power corresponding to the power of the set focus characteristic threshold (e.g., the power used in capturing the frame that provided the highest focus characteristic).

Optionally, an initial focal plane setting in an automatic focusing process is associated with a user-set focus. For example, camera 302 may include an input device 340 (e.g., a button, a slider, a knob, a rotatable ring, a touch input device, etc.) that a user may use to set a focus of the camera 302, and an automatic focusing process may set an initial focal plane setting based on the user-set focus.

The CCU 120 may implement different modes for automatic focusing, such as a continuous focusing mode and a trigger focus mode. The continuous focusing mode may include the CCU 120 continuously executing the automatic focusing process (e.g., in real time) to keep the video in focus. In the trigger focus mode, the CCU 120 may use the automatic focusing process in response to a user request to focus (e.g., a user press of a button of the camera 104).

Returning to FIG. 2, method 200 may include, at step 204, generating focus maps for at least some of the video frames of the series of video frames. Step 204 may be performed, for example, by image processing unit 122 of system 100. Focus maps may be generated for each frame of a series of video frames received by the image processing unit 122 or may be generated for a subset of the series of video frames. For example, for a given frame rate of the series of video frames, focus maps may be generated for a fraction of the frame rate, such as one-half, one-third, one-quarter, one-tenth, or any other desired fraction.

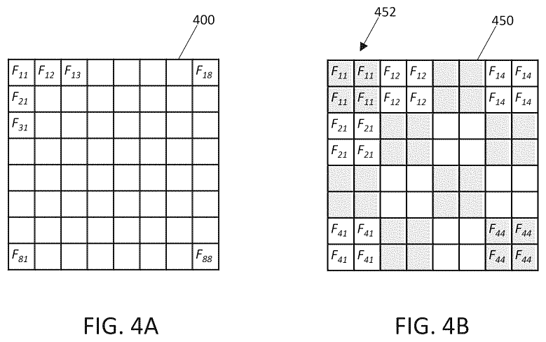

In general, a focus map for a given video frame is generated by determining focus metric values for different locations of the video frame based on pixel data of the video frame. A focus metric value for a given location of a frame may be an estimate of contrast associated with the location. Each focus map may include an array of focus metric values corresponding to estimates of contrast in a plurality of locations of a respective video frame. The size of the array may correspond to the pixel size of the video frame such that the array includes a focus metric value corresponding to each pixel of a given video frame. A focus metric value may be calculated uniquely for each pixel in the video frame. Alternatively, regional focus metric values may be generated such that the focus metric values corresponding to the pixels of a given region are the same. To illustrate these alternatives, FIG. 4A includes a representation of a focus map 400 generated for a frame of 8×8 pixels in which a focus metric value Fis calculated uniquely for each pixel of the 8×8 frame, and FIG. 4B illustrates a focus map 450 generated for a frame of 8×8 pixels in which focus metric values F are calculated for regions of 4×4 pixels such that the focus metric values for the pixels of a given region are the same (e.g., the focus metric values of each pixel location of region 452 are the same F₁₁).

A focus map may be generated based on a luminance channel of a given video frame. For example, raw video frame data may be converted to luminance and chroma channels and the focus map may be generated based on only the luminance channel (not the chroma channels). In some examples, a given video frame includes a plurality of color channels (e.g., red, green, and blue), and a focus map is generated based on fewer than all of the color channels. For example, a focus map may be generated based on only one of the color channels, such as just the green color channel (e.g., the green color channel may be a sufficiently close approximation of the luminance channel).

Step 204 may include applying at least one smoothing filter to smooth variation. The at least one smoothing filter may include a spatial filter for smoothing spatial variation across the dimensions of a focus map. The at least one smoothing filter may include a temporal filter that smooths variation between images such that a filtered focus map is a function of one or more other focus maps generated before and/or after the given focus map. The at least one smoothing filter may be a spatiotemporal filter that spatially and temporally filters a focus map. Examples of suitable filtering functions that can be used for temporal filtering and/or spatial filtering include Gaussian filters, mean filters, median filters, and bilateral filters. Applying at least one smoothing filter may include generating an initial focus map and applying at least one filter to the initial focus map to generate a filtered focus map. In the following steps, reference to “focus map” encompasses focus maps that are filtered and focus maps that are not filtered.

At step 206 of method 200, extended depth of field video frames are generated by combining image data from video frames of the series of video frames based on the focus maps generated at step 204. For example, after generating focus maps for multiple video frames in a series of video frames, the image processing unit 122 may combine at least some of the image data from at least some of the frames of the series of video frames for which focus maps were generated based on the focus maps for those video frames. Image data from any number of frames may be combined, including as few as two frames. In general, step 206 involves combining image data from frames such that more of the field of view is in-focus in the combined image relative to each individual frame. For example, given a first frame in which a first portion of the field of view is within the range of in-focus depths of the first frame (i.e., the first portion is in-focus) and a second frame in which a second portion of the field of view is within the range of in-focus depths of the second frame (i.e., the second portion is in-focus), a combined frame may include the in-focus first portion from the first frame and the in-focus second portion from the second frame such that, in the combined frame, both the first portion and the second portion of the field of view are in-focus. In general, the more frames captured using different focal plane settings that are combined, the greater the depth of field but the more latency. As such, the number of frames that are combined may be enough to provide as much of the scene in-focus without latency being noticeable (or unacceptably noticeable) to a viewer.

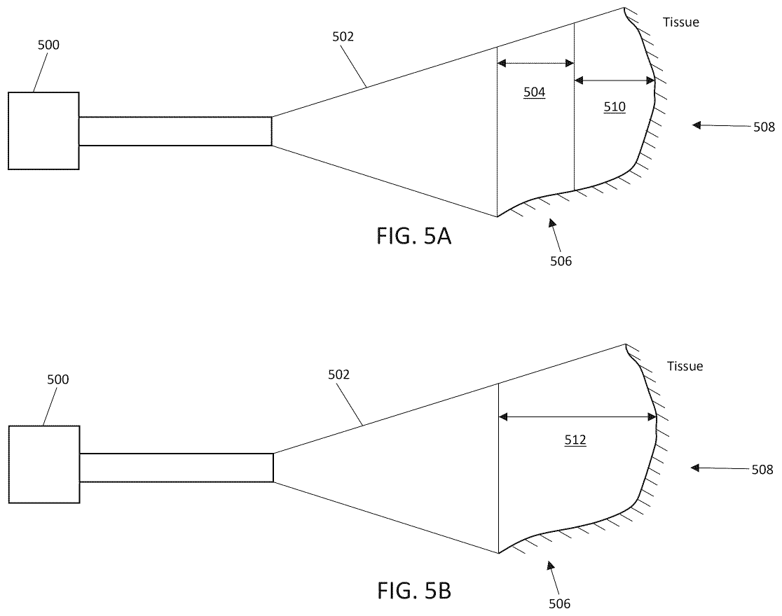

FIGS. 5A and 5B are schematic illustrations of the combining of endoscopic video frames of a target region using different focal plane settings to obtain an extended depth of field video frame according to method 200. In the example illustrated in FIG. 5A, an endoscopic imager 500 having a field of view 502 is used to capture a series of video frames of a region of tissue of interest. A focal plane setting of the endoscopic imager 500 can be set such that the endoscopic imager 500 has a first range of in-focus depths 504. A first portion 506 of the tissue being imaged is within the first range of in-focus depths 504 and, as such, is in-focus in frames captured when the endoscopic imager 500 is set to have the first range of in-focus depths 504. A second portion 508 of the tissue being imaged is not within the first range of in-focus depths 504 and, as such, is out-of-focus in frames captured when the endoscopic imager 500 is set to have the first range of in-focus depths 504. The focal plane setting of the endoscopic imager 500 can be set such that the endoscopic imager 500 has a second range of in-focus depths 510. The first portion 506 of the tissue being imaged is not within the second range of in-focus depths 510 and, as such, is out-of-focus in frames captured when the endoscopic imager 500 is set to have the second range of in-focus depths 510. The second portion 508 of the tissue being imaged is within the second range of in-focus depths 510 and, as such, is in-focus in frames captured when the endoscopic imager 500 is set to have the second range of in-focus depths 510.

A frame captured with the endoscopic imager 500 set to have the first range of in-focus depths 504 and a frame captured with the endoscopic imager 500 set of have the second range of in-focus depths 510 may be combined to produce an extended depth of field image having an extended range of in-focus depths 512, as illustrated in FIG. 5B. In such an extended depth of field image, both the first portion 506 of the tissue and the second portion 508 of the tissue are in-focus.

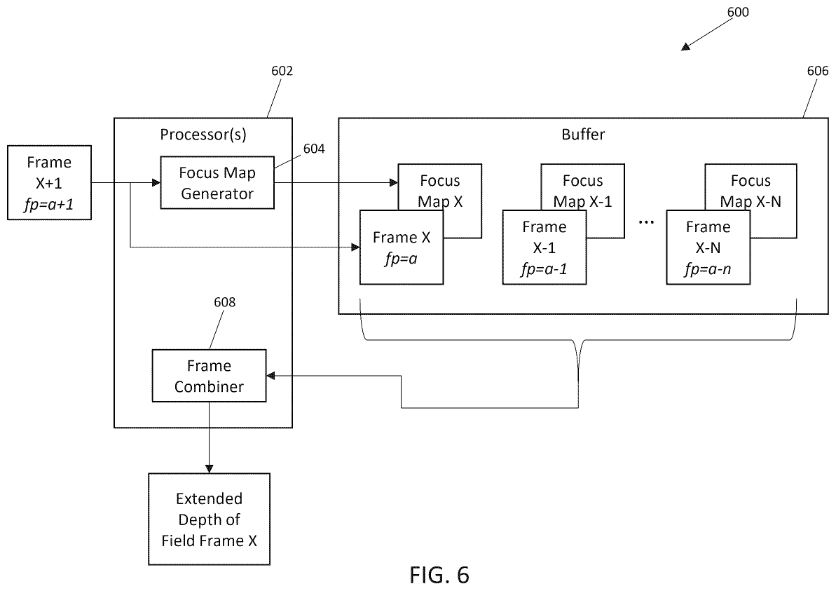

FIG. 6 is a functional block diagram of an exemplary image processing unit 600, which is an example implementation of image processing unit 122 of system 100. FIG. 6 illustrates how an image processing unit, such as image processing unit 122, may be configured to combine frames to generate extended depth of field frames. The functional components illustrated in FIG. 6 can be implemented as software, hardware, or a combination of software and hardware. At least one processor 602 of the image processing unit 122 receives video frames (e.g., from CCU 120). The processor 602 includes a focus map generator 604 that processes a received video frame to generate a focus map for the video frame. The processor 602 may store a video frame and its associated focus map in a buffer 606 of an associated memory. The processor 602 includes a frame combiner 608 that retrieves multiple frames from the buffer 606 and combines image data from the frames based on their focus maps to generate an extended depth of field frame. In the illustrated example, image data from frames X through X−N, each of which was captured with a different focal plane setting (fp=a through fp=a−n), are combined into an extended depth of field frame X based on focus maps of the respective frames (focus map X through focus map X−N). A frame may be removed from the buffer 606 (e.g., the oldest frame X−N may be removed from the buffer) and a newly received frame (e.g., frame X+1) and newly generated focus map (e.g., generated from frame X+1) may be stored in the buffer. The number of frames and focus maps stored in the buffer 606 may correspond with the number of frames that contribute image data that are combined to generate the extended depth of field frame. Alternatively, the number of frames and focus maps stored in the buffer 606 may be greater than the number of frames that contribute image data that are combined into a given combined frame, which may enable dynamic adjustment of the number of frames that contribute to a given combined frame. The number of frames that are stored in the buffer 606 and contribute image data to be combined can be selected to balance the degree of increased depth of field in the image with acceptable latency. The number of frames that are stored in the buffer 606 and contribute image data to be combined may be adjusted dynamically based on what is occurring in the field of view. For example, where the field of view does not have highly varying depth, fewer frames may be needed to provide the depth of field needed for the field of view and, thus, fewer frames may be stored in the buffer 606 and contribute image data to be combined, and conversely, where the field of view has highly varying depth, more frames may be needed to provide the depth of field needed for the field of view and, thus, more frames may be stored in the buffer 606 and contribute image data to be combined.

The frame combiner 608 may combine image data from frames in a number of different ways. In some examples, the frame combiner 608 converts each focus map into an array of weights corresponding to the array of focus metric values of the focus map. A number of different weighting functions could be used. For example, the weights could be a normalization of the focus metric values (e.g., normalized in the range of 0 to 1), or a sigmoid function could be used to heavily favor pixel values corresponding to high focus metric values while heavily minimizing the contribution of pixel values corresponding to low focus metric values.

The weights for a given frame may then be used to combine pixel values of the frames into a combined frame such that highly weighted portions of a given frame contribute to a greater degree to the corresponding portions of the combined frame. In general, each pixel value in the combined frame is a function of the corresponding pixel values of the frames that are combined and the corresponding weights for those frames, according to the following:

$${p}_{r , c}^{′} = f ( {p}_{r , c}^{1} , {w}_{r , c}^{1} , … , {p}_{r , c}^{n} , {w}_{r , c}^{n} )$$

where,

$${p}_{r , c}^{′}$$

is the pixel value for row r, column c of the combined frame,

$${p}_{r , c}^{1}$$

is the pixel value for row r, column c of original frame 1, and

$${w}_{r , c}^{1}$$

is the weight corresponding to row r, column c of original frame 1.

The weights for a given frame may be generated using one or more spatial and/or temporal filters to smooth variability. The filter(s) may be applied to focus maps prior to converting the focus maps into weight matrices and/or may be applied to the weight matrices.

Optionally, frame combiner 608 is configured to select frames to use in generating an extended depth of field frame based on whether the frames sufficiently contribute to increasing depth of field. For example, where frame X and frame X−1 are insufficiently different in terms of what portions or how much of a field of view are in focus, frame combiner 608 may use one of the frames but not the other in generating the extended depth of field frame. Frame combiner 608 may compare focus maps to determine whether a given frame should be used in generating an extended depth of field frame. For example, frame combiner 608 may compare focus map X and focus map X−1 to determine whether frame X and frame X−1 are insufficiently different in terms of what portions or how much of a field of view are in focus. The frame combiner 608 may compare a focus map of a given frame to focus maps of any frames in the buffer 606 to determine whether the given frame sufficiently contributes to extending the depth of field relative to any of the frames in the buffer 606. In comparing focus maps of different frames, the frame combiner 608 may determine whether one or more corresponding regions of different focus maps are sufficiently different (e.g., the differences between focus metric values for a given region exceed a predetermined threshold). Optionally, a determination of whether a given frame sufficiently contributes to increasing depth of field is made prior to storing the frame in the buffer 606. For example, processor(s) 602 may be configured to compare a focus map generated for frame X+1 to focus maps X to X−N in buffer 606 to determine whether frame X+1 sufficiently contributes to increasing depth of field and, thus, whether to add frame X+1 to the buffer 606.

Optionally, combining frames according to step 206 includes frame-to-frame registration to compensate for motion of the camera and/or target. Any suitable motion compensation algorithm can be used to determine motion between frames and to correct for the motion by shifting pixels of one frame to align with corresponding pixels of one or more other frames. In some variations, no frame-to-frame registration is used, such as when the frame rate is sufficiently high such that any motion between images is negligible or acceptable.

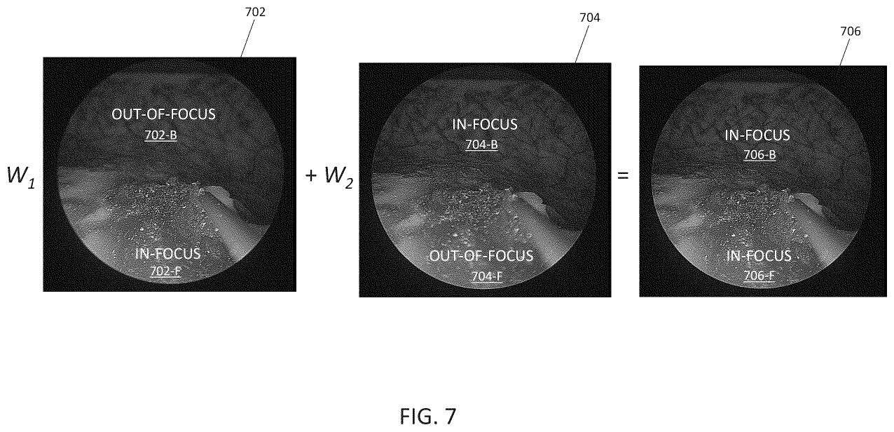

FIG. 7 illustrates an example of the combination of image data from two endoscopic imaging frames captured with different focal plane settings into an extended depth of field frame. Frame 702 was captured with a first focal plane setting that resulted in the foreground 702-F of the imaged region being within the range of in-focus depths but not the background 702-B (the foreground 702-F is more in-focus than the background 702-B). Frame 704 was captured with a second focal plane setting that resulted in the background 704-B of the imaged region being within the range of in-focus depths but not the foreground 704-F (the background 704-B is more in-focus than the foreground 704-F). Image data from frame 702 and frame 704 were combined into combined frame 706 that has an extended depth of field. Image data from frame 702 and frame 704 were combined using weights W₁ and W₂ such that the foreground 706-F of combined frame 706 is dominated by the foreground 702-F of frame 702 and the background 706-B of combined frame 706 is dominated by the background 704-B of frame 704, thus resulting in both the foreground 706-F and background 706-B being in-focus.

Frames may be combined into an extended depth of field frame, according to step 206 of method 200, based on content of the frames, such as determined by image processing unit 122 of FIG. 1. Frames may be segmented into one or more regions based on their content. For example, with reference to FIG. 2, method 200 may include optional step 205 in which video frames are segmented. Frames may be combined into an extended depth of field frame in step 206 based on the results of the segmentation. For example, a region that includes content that is desirable to include in the depth of field, such as a region of tissue, may be identified in step 205 via an image segmentation process, and frames may be combined in step 206 in a manner that prioritizes focus of that region. Prioritizing a particular region when combining frames into an extended depth of field frame may include, for example, heavily weighting pixel values for that region in a frame that provides the best contrast.

Frames may be segmented using any suitable image segmentation process. An exemplary image segmentation process may segment a frame based on color. For example, frames may be segmented into red regions and non-red regions (e.g., by comparing the red component of RGB pixel values a threshold value), with red regions being associated with tissue that is desirable to include in the depth of field of the extended depth of field image. The same segmented red region(s) in a series of frames may be combined in an extended depth of field frame in a way that maximizes the focus of the segmented red region.

An exemplary image segmentation process may segment a frame based on brightness. For example, very bright regions may be associated with reflection from surgical instruments, and it may be undesirable to combine frames in a way that emphasizes those regions. As such, frames may be combined into an extended depth of field image in a manner that ignores or de-emphasizes segmented regions associated with the surgical instrument.

An exemplary image segmentation process may include using a machine learning model to perform image segmentation, such as semantic segmentation. A machine learning model may be trained to segment various different types of content that may be captured during a medical procedure, such as tissue, different types of tissue, instruments, etc. Frames may be combined into an extended depth of field frame in a way that prioritizes the focus of segmented regions that should be in-focus, such as tissue, over segmented regions that do not need to be in focus, such as instruments. For example, an image processing system may determine, for each segment, which frame provides the better focus and may use the segment of that frame in the extended depth of field frame along with one or more segments of one or more other frames.

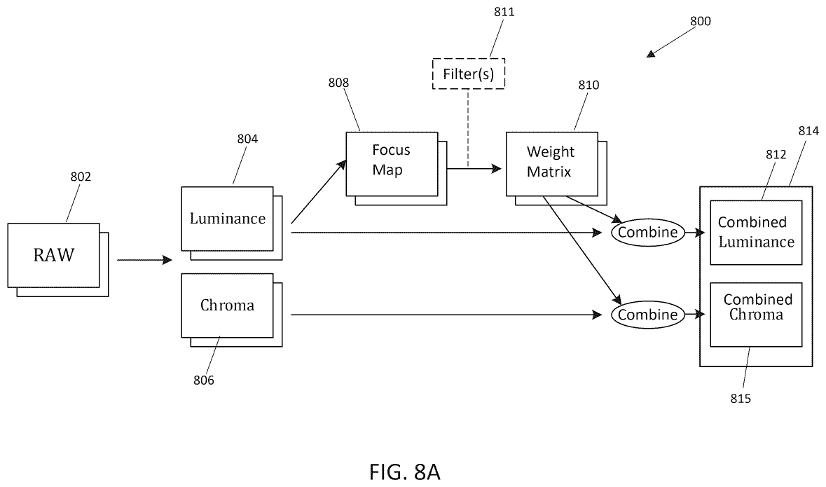

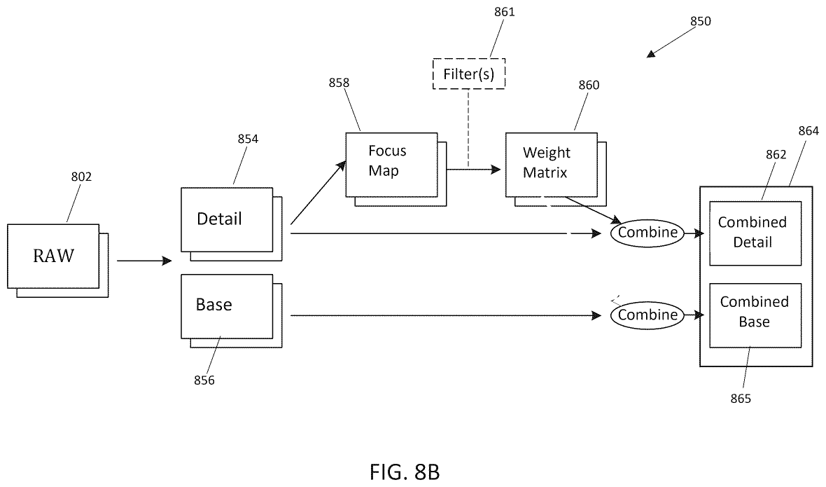

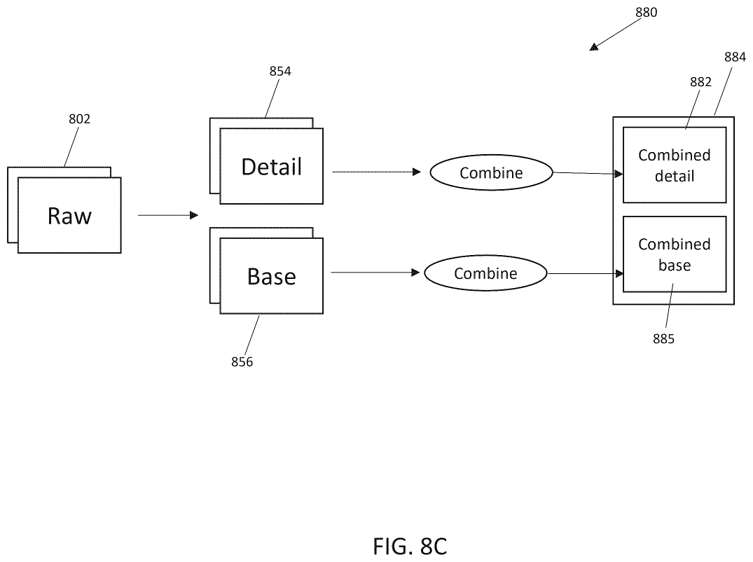

Focus maps may be generated from raw video frame data or may be generated from data generated from raw video frame data. For example, raw pixel data may be converted into luminance and chroma channels, and the focus maps may be generated based on the luminance channel. FIG. 8A illustrates an exemplary method 800 for generating a combined image having an extended depth of field, according to method 200, in which focus maps are generated based on the luminance channel of video frames. Method 800 may be performed, for example, by image processing unit 122 of system 100 of FIG. 1. The raw data of a plurality of video frames 802 (e.g., red, green, and blue pixel data) may be converted into respective luminance channels 804 and chroma channels 806 on a frame-by-frame basis. A focus map 808 may be generated from each luminance channel 804 (e.g., at step 204 of method 200). Each focus map 808 may be converted into a weight matrix 810 (with or without applying one or more filters 811, such as one or more spatial filters and/or one or more temporal filters). The weight matrices 810 may then be used to combine data of the luminance channels 804 of multiple frames into a luminance channel 812 of a combined frame 814 (e.g., at step 206 of method 200). The weight matrices 810 may be used to combine data of the chroma channels 806 into chroma channels 815 of the combined frame 814. Alternatively, the chroma channels 806 of the combined image may be chroma channels of the original image, which may be acceptable since the chroma channels may include significantly less detail information than the luminance channel and which may be beneficial in reducing the processing power required to generate the combined frame 814. Optionally, the weight matrices 810 may be used to combine raw data of the plurality of video frames 802.

A process similar to that of FIG. 8A may be applied using other types of image decomposition. For example, a suitable image decomposition technique can be used to convert raw frame data (red, green, and blue pixel data) or converted frame data (e.g., a luminance channel) into a low spatial frequency layer (often referred to as a base layer) and a high spatial frequency layer (often referred to as a detail layer), and the focus maps may be generated from the detail layer. The focus maps or weight matrices generated from the focus maps may be used to combine the detail layer and/or the base layer. For example, the detail layers of multiple frames may be combined based on the contrast in the detail layers as reflected in the focus maps (e.g., a weighted sum) and the base layers may be combined by averaging.

An example of a process similar to FIG. 8A but using high and low spatial frequency decomposition is illustrated in FIG. 8B. In method 850 of FIG. 8B (which may be performed, for example, by image processing unit 122 of system 100 of FIG. 1), the raw data of a plurality of video frames 802 (e.g., red, green, and blue pixel data) may be converted into respective detail layer 854 and base layer 856 on a frame-by-frame basis. A focus map 858 may be generated from each detail layer 854 (e.g., at step 204 of method 200). Each focus map 858 may be converted into a weight matrix 860 (with or without applying one or more filters 861, such as one or more spatial filters and/or one or more temporal filters). The weight matrices 860 may then be used to combine data of the detail layer 854 of multiple frames into a combined detail layer 862 (e.g., at step 206 of method 200). The base layers 856 may be combined into a combined base layer 865, such as by averaging. The combined detail layer 862 and combined base layer 865 may be converted into a combined frame 864 having an extended depth of field.

In some variations, a detail layer of a frame is, itself, the focus map for the frame (e.g., in step 204 of method 200, the focus maps are generated by converting the frames into details layers). The extended depth of field frame may be generated (e.g., in step 204 of method 200) by combining the detail layers of multiple frames (e.g., summing the detail layers) into a combined detail layer, combining the base layers of the multiple frames (e.g., averaging the base layers) into a combined base layer, and converting the combined detail layer and combined base layer into an extended depth of field frame. FIG. 8C illustrates an exemplary method 880 that may be performed, for example, by image processing unit 122 of system 100 of FIG. 1 in which the detail layer of a frame is the focus map for the frame. The raw data of a plurality of video frames 802 (e.g., red, green, and blue pixel data) may be converted into respective detail layer 854 and base layer 856 on a frame-by-frame basis. The detail layers 854 of multiple frames may be combined into a combined detail layer 882, such as by summing the detail layers 854 of the multiple frames. The base layers 856 of multiple frames may be combined into a combined base layer 885, such as by summing the base layers 854 of the multiple frames. The combined detail layer 882 and combined base layer 885 may be converted into a combined frame 884 having an extended depth of field.

In some examples in which the raw frame data includes red, green, and blue channels, the focus maps may be generated from fewer than all of the channels. For example, the focus maps may be generated only from the green channel, which may be suitable since the green channel typically captures more human-perceivable detail than the other channels. Focus maps generated from just the green channel may be used to combine only the green channel or may be used to combine other channels, as well.

Returning to FIG. 2, at step 208, the extended depth of field video frames generated at step 206 are displayed, such as on display 124 of system 100. The extended depth of field video frames may be displayed continuously at a rate that is sufficient for the extended depth of field video frames to appear to the viewer to be displayed in real time. The extended depth of field video frames may be generated and displayed at the same frame rate as the video frames are captured but delayed in time according to the number of frames stored in the buffer and the time required to generate the focus maps and combine the video frames. Alternatively, the extended depth of field video frames may be generated at a lower rate than the rate used to capture frames and may be displayed at the same rate as a display rate. For example, frames may be captured at 120 frames per second and extended depth of field video frames may be generated and displayed at 60 frames per second.

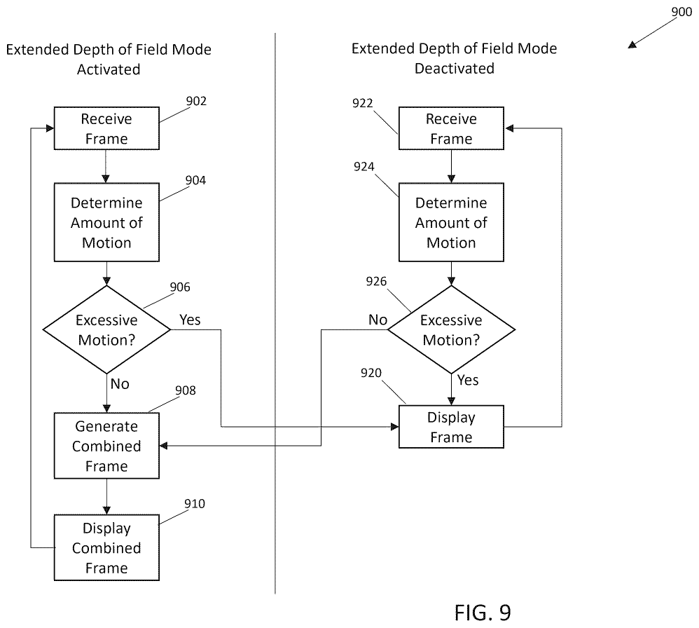

The combining of image data from videoframes into an extended depth of field frame may be performed in an extended depth of field mode that can be manually or automatically enabled and disabled. The extended depth of field mode may be automatically enabled and disabled based on an amount of motion captured in a series of video frames. Frames generated with excessive motion may have sufficiently different fields of view that combining image data from them may produce undesired results. As such, the extended depth of field mode may be exited and/or deactivated when the motion is excessive. FIG. 9 illustrates an exemplary method 900 for automatically entering and exiting an extended depth of field mode based on the amount of motion in a series of video frames. Method 900 may be performed, for example, by image processing unit 122 of system 100.

Method 900 will be described from the perspective that the extended depth of field mode is activated, which may be a default mode. At step 902, a frame is received by the image processing unit. At step 904, the frame may be compared to one or more previously received frames stored in a buffer to determine an amount of motion of the field of view between the frames. Any suitable motion estimation algorithm may be used. For example, a similarity score may be calculated from a pair of frames, and the similarity score may be used as an estimate of the amount of motion. At step 906, the amount of motion determined at step 904 is compared to one or more predetermined thresholds to determine whether the amount of motion is excessive. If the amount of motion is not excessive, then a combined frame is generated at step 908 and displayed at step 910. The method then proceeds back to step 902 for a next frame.

If the amount of motion is determined to be excessive at step 908, the extended depth of field mode may be deactivated, and the received frame is displayed at step 920. At step 922, a next frame is received. At step 924, the frame may be compared to one or more previously received frames stored in a buffer to determine an amount of motion of the field of view between the frames. At step 926, the amount of motion determined at step 924 is compared to one or more predetermined thresholds to determine whether the amount of motion is excessive (the same threshold(s) used in step 906 may be used in step 926). If the amount of motion continues to be excessive, then the frame is displayed at step 920. If the amount of motion is not excessive, then the extended depth of field mode may be reactivated, and method 900 may return to step 902.

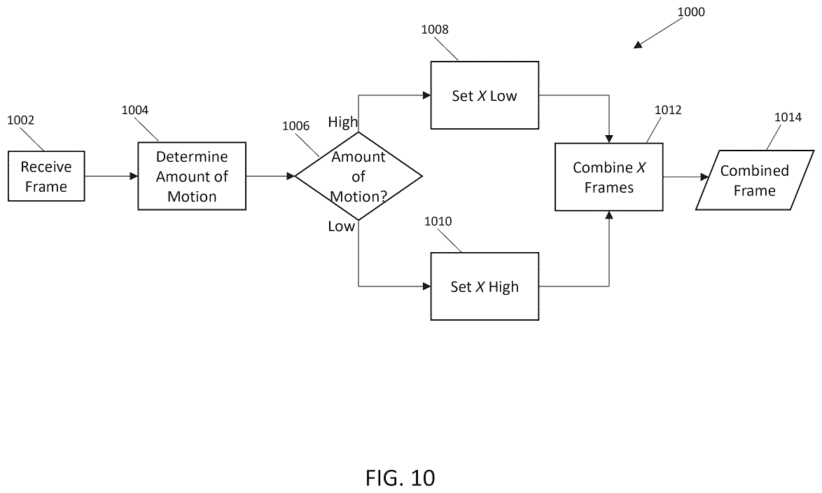

Rather than deactivating the extended depth of field mode when motion is excessive, the number of frames that are combined into an extended depth of field frame may be reduced. FIG. 10 illustrates an exemplary method 1000 for adjusting the number of frames used to generate each extended depth of field frame. Method 1000 may be performed, for example, by image processing unit 122 of system 100. A video frame is received at step 1002 and the video frame is analyzed at step 1004 to determine an amount of motion in similar fashion to step 1004 of method 1000. At step 1006, the amount of motion determined at step 1004 is compared to one or more threshold values to categorize the amount of motion based on one or more predetermined categories. The categories may be as few as two—e.g., no excessive motion versus excessive motion—or any number above two—e.g., low, medium, and high motion. The example illustrated in FIG. 10 shows a binary decision between a low motion category and a high motion category. If the amount of motion is determined at step 1006 to be high, then at step 1008, a parameter X is set to a low value. If the amount of motion is determined at step 1006 to be low, then at step 1010, the parameter X is set to a high value. Then, at step 1012, a number of frames corresponding to the value of parameter X are combined to generate a combined frame 1014. Thus, sufficiently high motion results in fewer frames being combined into a combined frame, and sufficiently low motion results in more frames being combined into a combined frame. Parameter X can take on more values where, for example, more categories of motion are determined. For example, each of low, medium, and high levels of motion can be associated with three different values of parameter X.

Optionally, method 1000 and method 900 may be combined. For example, method 1000 may be performed while in the extended depth of field mode to account for different amounts of motion that still permit frames to be combined. If the amount of motion exceeds an upper threshold, as determined at step 1006, then the extended depth of field mode may be deactivated as explained above with reference to method 900 until motion sufficiently reduces.

Any of methods 200, 800, 900, and 1000 may generate combined frames by accounting for motion between frames. For example, step 908 of method 900 and/or step 1012 of method 1000 may include compensating for motion when combining frames. Any suitable motion compensation algorithm may be used to combine frames.

As noted above, system 100 may be a multi-modality imaging system that can capture video frames using multiple different modalities. For example, the camera 104 may be configured to capture both reflected visible light frames and fluorescence light frames. The reflected light frames and fluorescence light frames may be captured in an alternating manner and combined for display, such as in the form of an overlay of fluorescence information on a reflected light frame. When generating images in a multi-modality mode, frames from one or both imaging modalities may be combined into extended depth of field frames according to any of methods 200, 800, 900, and 1000. For example, image processing unit 122 may use method 200 to generate extended depth of field reflected light frames by combining image data from reflected light frames and/or to generate extended depth of field fluorescence light frames by combining image data from fluorescence light frames. An extended depth of field reflected light frame may be combined with a corresponding fluorescence frame or with an extended depth of field fluorescence frame. Optionally, focus maps generated from frames of one imaging modality may be used to generate extended depth of field frames for another imaging modality. For example, focus maps generated for reflected light frames may be used to combine image data from fluorescence light frames.

According to an aspect, an imaging system may be configured to use different focal plane settings for different imaging modalities when operating in a multi-modality imaging mode such that focal plane settings used for capturing frames of one imaging modality are different than focal plane settings used for capturing frames of another imaging modality. The different focal plane setting can be used to account for differences in the propagation of different wavelengths of light associated with the different imaging modalities. A multi-modality imaging mode can include capturing frames for one imaging modality in an alternating fashion with frames of another imaging modality and changing focal plane settings accordingly. Different imaging modality frames may be combined into combined frames and displayed to a user as multi-modality video. For example, image data from fluorescence frames and reflected white light frames may be combined into frames in which the fluorescence signal is overlayed on the reflected white light frame.

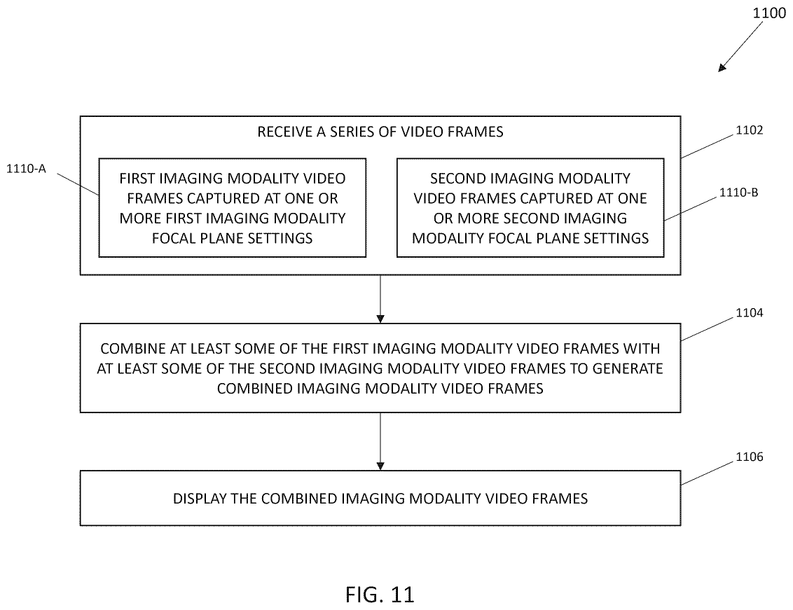

FIG. 11 illustrates an exemplary method 1100 for multi-modality imaging using different focal plane settings for different modalities. Method 1100 may be performed, for example, by system 100 of FIG. 1. At step 1102, a series of video frames is received at a computing system, such as image processing unit 122 of system 100. The series of video frames may be received, for example, from CCU 120. The series of frames includes first imaging modality video frames 1110-A captured by a camera (e.g., camera 104 of system 100) at one or more first focal plane settings used for a first imaging modality and second imaging modality video frames 1110-B captured by the camera at one or more second focal plane settings used for a second imaging modality. The first and second imaging modality frames may be received in alternating fashion. For example, a first imaging modality frame may be received, followed by a second imaging modality frame, followed by another first imaging modality frame, and so on. The different imaging modalities may be associated with imaging different wavelengths of light. For example, a first imaging modality may be a reflected light imaging modality in which tissue of interest is imaged using light (e.g., broad-band light, such as white light) that is reflected from tissue of interest, and a second imaging modality may be a fluorescence imaging modality in which fluorescence light (e.g., infrared light, such as near infrared or short-wave infrared) is emitted by the tissue of interest. A light source, such as light source 108, may be controlled to provide the light required for each imaging modality in an alternating fashion. For example, with reference to FIG. 1, the light source may be controlled to provide white light via the one or more visible light emitters 110 for capturing a first imaging modality frame and to provide fluorescence excitation light via the one or more excitation light emitters 112 for capturing a second imaging modality frame.

The camera may be controlled such that different focal plane settings are used when capturing the different imaging modality frames. As such, the focal plane settings may be switched between successive frames in an alternating fashion. For example, with reference to FIG. 3, the liquid lens 310 may be controlled to set a first focal plane setting, a first imaging modality frame may be captured at this first focal plane setting, the liquid lens 310 may be controlled to set a second focal plane setting, and a second imaging modality frame may be captured at this second focal plane setting. The different focal plane settings used for the different imaging modalities may account for differences in the kind of light being used for each imaging modality. For example, a first focal plane setting may be better for imaging broad-band visible light for reflected light imaging, and a second focal plane setting may be better for imaging infrared light for fluorescence imaging. Using different focal plane settings for different imaging modalities may improve the quality of the frames of each of the imaging modalities and/or may provide a larger design envelope for the optical elements of the camera, enabling a wider range of different configurations of optical elements to be used. This could be advantageous, for example, in enabling an endoscopic camera head to be used with a greater variety of endoscopes.

Step 1102 can include an image processing unit (e.g., image processing unit 122 of system 100) controlling a light source (e.g., light source 108) and a camera (e.g., camera 104) synchronously to obtain the different imaging modality frames in an alternating fashion. For example, the image processing unit may control the timing that the light source switches from providing light for one imaging modality to providing light for another imaging modality and may synchronously control the timing that the camera 104 switches its focal plane setting from one used for the first imaging modality to one used for the second imaging modality. Such switching may occur, for example, at a frame rate of the camera 104.

At step 1104, image data from at least some of the first imaging modality video frames are combined with image data from at least some of the second imaging modality video frames to generate combined imaging modality video frames. For example, image data from reflected visible light frames and fluorescence light frames may be combined into frames that provide the fluorescence signal as an enhanced color (e.g., green) in the visible light frame. At step 1106, the combined imaging modality video frames are displayed. For example, the image processing unit 122 may output the combined imaging modality video frames to display 124 of system 100.

Different focal plane settings may be used for different frames of a given imaging modality. Frames of a given imaging modality may be captured using different focal plane settings and combined to generate extended depth of field frames. For example, one or more steps of method 200 of FIG. 2 and/or method 600 of FIG. 6 may be used to generate extended depth of field frames for one or more of a plurality of imaging modalities. Different ranges of focal plane settings may be used for the different imaging modalities such that the different focal plane settings used for generating extended depth of field frames for a particular imaging modality are within a first range and the focal plane settings used for generating extended depth of field frames for another imaging modality are within a second range that is different from the first range (the different ranges may or may not overlap).

Optionally, one or more focal plane settings used for the first imaging modality and/or one or more focal plane settings used for the second imaging modality may be determined dynamically based on at least one focus estimate. A frame of a given imaging modality may be analyzed (e.g., by image processing unit 122 of system 100) to generate a focus estimate that estimates a relative focus of the frame, and a focal plane setting may be adjusted for capturing the next frame of the given imaging modality to improve the focus. For example, an automatic focusing algorithm may be used to search for the focal plane setting for a given imaging modality that provides suitable focus. Focus estimates may be generated for frames of multiple imaging modalities and may be used to set the focal plane settings for the multiple imaging modalities. For example, an automatic focusing algorithm can be used for each of multiple imaging modalities to determine the best focal plane setting for each imaging modality.

Optionally, the aperture of the camera may be an adjustable aperture that may be adjusted for different imaging modalities. For example, camera 302 of FIG. 3 may include an adjustable aperture 350 that may be controlled by controller 314 to change the aperture size. For example, the aperture size may be increased when capturing fluorescence frames to increase the amount of light from the scene that reaches the imaging sensor, and the aperture size may be decreased when capturing reflected light frames so as to not saturate the image sensor(s) and to increase the depth of field of a given frame. Adjustable aperture sizes may be used when capturing frames according to just a single imaging modality, such as to account for different working distances. Regardless of whether aperture size is adjusted when operating in a single imaging modality or multiple imaging modalities, focal plane settings of the camera may be automatically adjusted at least in part to account for different aperture sizes.

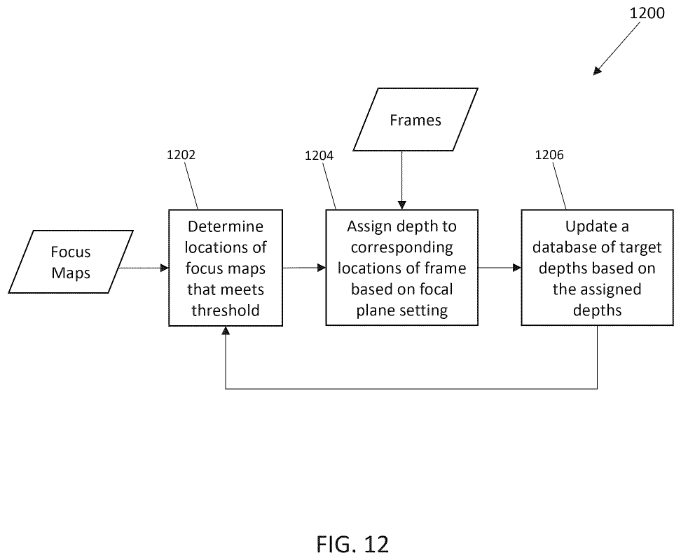

Focus maps generated according to any of the methods described herein can be used for purposes other than combining image data from different frames into extended depth of field frames. For example, focus maps may be used to generate estimates of target depth. Estimates of target depth may be useful, for example, in generating a three-dimensional model of a target, generating three-dimensional measurements between various locations of the target, and/or any other purposes in which three-dimensional information about the target is desired. FIG. 12 illustrates an exemplary method 1200 for generating estimates of target depth using focus maps. Method 1200 may be performed, for example, by image processing unit 122 of system 100 of FIG. 1. Method 1200 may utilize predetermined depths associated with different focal plane settings of a camera. The optical components of the camera can be characterized to determine the depths of objects that are in-focus for a given focal plane setting. With this information, the portions of a field of view that are in-focus in a given frame—i.e., are within the range of in-focus depth—can be determined and assigned the predetermined depth or range of depths associated with the focal plane setting used for capturing the frame. Different frames captured with different focal plane settings can be used to obtain the depths of different portions of the field of view.

Method 1200 may include, at step 1202, determining the locations of a focus map that meet a predetermined threshold associated with discriminating in-focus locations from out-of-focus locations of a frame generated using a first focal plane setting. The focus map can be a focus map generated at step 204 of method 200. The locations of the focus map that meet the predetermined threshold are locations that are more in-focus than locations of the focus map that do not meet the predetermined threshold.

At step 1204, at least one depth is assigned to locations of the frame (the frame corresponding to the focus map analyzed at step 1202) that correspond to the locations of the focus map determined at step 1202 based on a focal plane setting associated with the frame. The depth can be a value that was predetermined based on characterization of the camera when set to the focal plane setting. The camera may have been characterized at each of its focal plane settings to determine the range of distances (e.g., in millimeters or inches) from a predetermined location of the camera (e.g., the tip of an endoscope of an endoscopic camera, the imaging plane, or any other fixed location of the camera) that are within the range of in-focus depths of the camera at each focal plane setting. For each focal plane setting, objects that are within the range of in-focus depths associated with the focal plane setting will be in focus when the camera is set to the corresponding focal plane setting. The depth assigned to locations of a frame in step 1204 can be, for example, a median distance in the range of depths associated with the focal plane setting for the frame. Step 1204 can include assigning the depth associated with the focal plane setting for each pixel of the frame for which the corresponding location of the focus map meets the predetermined threshold.

At step 1206, a database of target depths is updated based on the depth assigned to the locations of the frame at step 1204. The database can include depths for different locations of the target, thus forming a “depth map.” For example, the database can include depths associated with a plurality of two-dimensional locations of the target (e.g., the −x and −y dimensions, where the depth is the −z dimension), and the database can be updated to include entries for each location of the frame to which the depth was assigned in step 1206. The two-dimensional locations may be in the same units as the depths (e.g., millimeters or inches) such that each database entry includes a three-dimensional location associated with the target in a desired unit.

Optionally, step 1206 can include determining the two-dimensional (e.g., −x and −y) locations associated with each depth in the same units as the depth. For example, the two-dimensional pixel locations corresponding to each depth can be used, along with the depth and predetermined knowledge about the camera, to determine the spatial location associated with each pixel location.

Optionally, the updating of the database with the target depths at step 1206 can take into account motion between frames. For example, a suitable motion compensation algorithm can be used to determine that the pixels captured in the frame are shifted relative to pixels of a previous frame and the shift can be compensated for when updating the database with the target depths.

Steps 1202 to 1206 can be repeated for subsequent frames (e.g., for each frame for which a focus map is generated), resulting in the database being populated with entries corresponding to multiple regions of the target that are at multiple different depths. The database can be considered a three-dimensional model of the target, with each entry being a three-dimensional location of the target, or the database can be used to generate a three-dimensional model of the target. Optionally, entries in the database are interpolated based on other entries such that a change in depth from one −x/−y location to another −x/−y location are smoothly varying.

Optionally, the focal plane settings used for capturing frames used in method 200 may be selected to provide a desired depth resolution of the focus map. For example, relatively small changes in focal plane settings may be used to provide an increased depth resolution for the focus map. Relatively small changes in focal plane settings may be used in an autofocusing process such that a depth map having a desired depth resolution can be generated based on frames captured during an automatic focusing process. Optionally, an autofocusing process may not re-use a previously used focal plane setting such that each frame used in the autofocusing process may contribute at least some new information to a depth map.

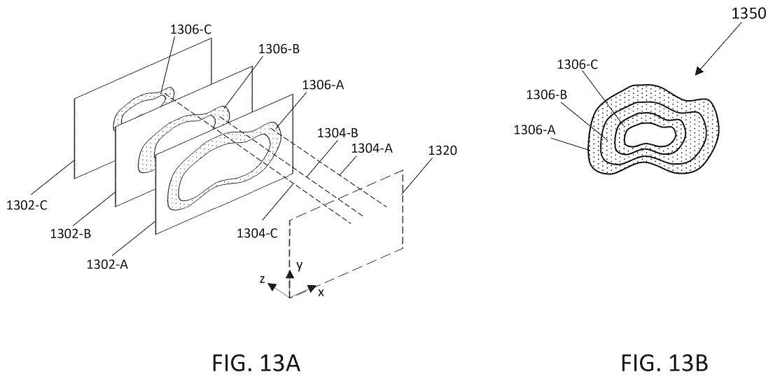

FIGS. 13A and 13B illustrate the application of method 1200 to a series of three video frames. Each frame 1302-A, 1302-B, and 1302-C is depicted at a different pre-determined depth from a predetermined plane 1320 (e.g., the imaging plane), the pre-determined depth corresponding to the range of in-focus depths of the focal plane setting for the frame (i.e., a distance in an optical axis direction from a predetermined plane 1320). Thus, frame 1302-A is associated with depth 1304-A, frame 1302-B is associated with depth 1304-B, and frame 1302-C is associated with depth 1304-C. Locations of each frame are identified as being in-focus based on the associated portions of the corresponding focus map meeting a predetermined focus threshold, as determined at step 1202. Frame 1302-A has the locations that are in-focus indicated by shaded region 1306-A, frame 1302-B has the locations that are in-focus indicated by shaded region 1306-B, and frame 1302-C has the locations that are in-focus indicated by shaded region 1306-C. The depths associated with each frame are assigned to the in-focus locations at step 1204. Thus, locations of region 1306-A are assigned depth 1304-A, locations of region 1306-B are assigned depth 1304-B, and locations of region 1306-C are assigned depth 1304-C. A database that includes the locations of the target and their assigned depths is represented graphically in FIG. 13B in the form of a depth map 1350.

In general, the narrower the depth of field of the camera, the higher the resolution of a depth map generated according to method 1200. A camera, such as camera 302 of FIG. 3, may include an adjustable aperture (e.g., adjustable aperture 350) that enables the depth of field to be narrowed by setting the aperture to a larger value. A larger aperture setting may be used in a depth map mode. The depth map mode may be initiated in response to a user request, such as a user request made via a user input provided to the image processing unit 122, to the CCU 120, directly to the camera 302, or to a user input device communicatively connected to the image processing unit 122, CCU 120, and/or camera 302. Additionally, or alternatively, the depth map mode may be initiated in response to initiation of a function that requires a depth map. For example, the user may enter a target measurement mode provided by the image processing unit 122 for generating measurements of the target based on the video frames, and the image processing unit 122 may control the camera 104 to switch to a depth map mode. In the depth map mode, the camera 104 may be controlled to adjust the aperture setting to a larger value, thereby providing a narrower depth of field for each focal plane setting. Since adjusting the aperture may affect the distances that are within the depth of field of a given focal plane setting (beyond narrowing the range of distances), the image processing unit 122 may store predetermined depths for each combination of aperture setting and focal plane setting.

Depth information generated based on focus maps associated with different imaging modalities may be combined to determine one or more characteristics of a region of tissue of interest that is not obtainable from either of the imaging modalities individually. For example, depth information determined based on one or more frames of a first imaging modality that is associated with imaging a surface of tissue may be combined with depth information determined based on one or more frames of a second imaging modality that is associated with imaging a feature below the surface of tissue to determine a depth of the feature below the surface of the tissue. The first imaging modality may be, for example, a reflected light imaging modality that images light reflected from the surface of a region of tissue, and the second imaging modality may be a fluorescence imaging modality that images fluorescence light emitted by fluorescence agent present in a feature located in below the surface of the region of tissue, such as one or more blood vessels. A depth of the surface of the region of tissue may be determined from one or more focus maps generated for one or more reflected light imaging modality frames and a depth of the feature located below the surface of the region of tissue may be determined from one or more focus maps generated for one or more fluorescence imaging modality frames. The depths can be compared to determine the depth of the feature below the surface of the region of tissue.

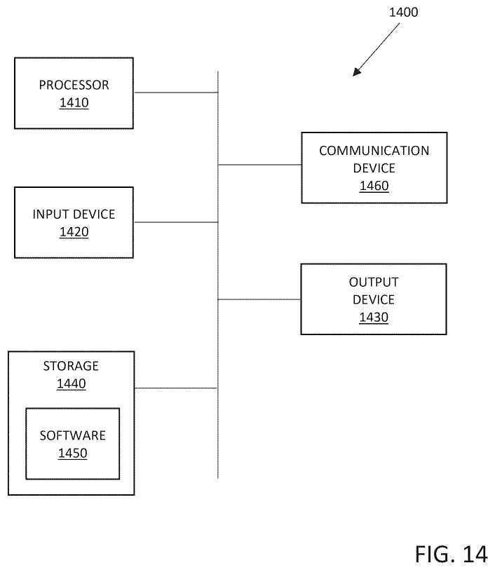

FIG. 14 illustrates an example of a computing system 1400 that can be used for one or more components of system 100 of FIG. 1, such as one or more of light source 108, camera control unit 120, camera 104, and image processing unit 122. System 1400 can be a computer connected to a network, such as one or more networks of a hospital, including a local area network within a room of a medical facility and a network linking different portions of the medical facility. System 1400 can be a client or a server. System 1400 can be any suitable type of processor-based system, such as a personal computer, workstation, server, handheld computing device (portable electronic device) such as a phone or tablet, or dedicated device. System 1400 can include, for example, one or more of input device 1420, output device 1430, one or more processors 1410, storage 1440, and communication device 1460. Input device 1420 and output device 1430 can generally correspond to those described above and can either be connectable or integrated with the computer.

Input device 1420 can be any suitable device that provides input, such as a touch screen, keyboard or keypad, mouse, gesture recognition component of a virtual/augmented reality system, or voice-recognition device. Output device 1430 can be or include any suitable device that provides output, such as a display, touch screen, haptics device, virtual/augmented reality display, or speaker.

Storage 1440 can be any suitable device that provides storage, such as an electrical, magnetic, or optical memory including a RAM, cache, hard drive, removable storage disk, or other non-transitory computer readable medium. Communication device 1460 can include any suitable device capable of transmitting and receiving signals over a network, such as a network interface chip or device. The components of the computing system 1400 can be connected in any suitable manner, such as via a physical bus or wirelessly.

Processor(s) 1410 can be any suitable processor or combination of processors, including any of, or any combination of, a central processing unit (CPU), field programmable gate array (FPGA), graphics processing unit (GPU), and application-specific integrated circuit (ASIC). Software 1450, which can be stored in storage 1440 and executed by one or more processors 1410, can include, for example, the programming that embodies the functionality or portions of the functionality of the present disclosure (e.g., as embodied in the devices as described above), such as programming for performing one or more steps of method 200 of FIG. 2, method 600 of FIG. 6, method 800 of FIG. 8A, method 850 of FIG. 8B, method 880 of FIG. 8C, method 900 of FIG. 9, method 1000 of FIG. 10, method 1100 of FIG. 11, and/or method 1200 of FIG. 12.

Software 1450 can also be stored and/or transported within any non-transitory computer-readable storage medium for use by or in connection with an instruction execution system, apparatus, or device, such as those described above, that can fetch instructions associated with the software from the instruction execution system, apparatus, or device and execute the instructions. In the context of this disclosure, a computer-readable storage medium can be any medium, such as storage 1440, that can contain or store programming for use by or in connection with an instruction execution system, apparatus, or device.

Software 1450 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate, or transport programming for use by or in connection with an instruction execution system, apparatus, or device. The transport computer readable medium can include, but is not limited to, an electronic, magnetic, optical, electromagnetic, or infrared wired or wireless propagation medium.

System 1400 may be connected to a network, which can be any suitable type of interconnected communication system. The network can implement any suitable communications protocol and can be secured by any suitable security protocol. The network can comprise network links of any suitable arrangement that can implement the transmission and reception of network signals, such as wireless network connections, Ethernet cables, fiber optic cables, DSL, or telephone lines.

System 1400 can implement any operating system suitable for operating on the network. Software 1450 can be written in any suitable programming language, such as C, C++, Java, or Python. In various examples, application software embodying the functionality of the present disclosure can be deployed in different configurations, such as in a client/server arrangement or through a web browser as a web-based application or web service, for example.

The foregoing description, for the purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the techniques and their practical applications. Others skilled in the art are thereby enabled to best utilize the techniques and various embodiments with various modifications as are suited to the particular use contemplated.

Although the disclosure and examples have been fully described with reference to the accompanying figures, it is to be noted that various changes and modifications will become apparent to those skilled in the art. Such changes and modifications are to be understood as being included within the scope of the disclosure and examples as defined by the claims. Finally, the entire disclosure of the patents and publications referred to in this application are hereby incorporated herein by reference.

1. A method for medical imaging comprising, at a computing system:
receiving a series of video frames of a region of tissue of a subject captured using different focal plane settings of a camera;
generating focus maps for at least some of the video frames of the series of video frames, wherein the focus maps are generated using at least one smoothing filter;
generating extended depth of field video frames by combining image data from video frames of the series of video frames based on the focus maps; and
displaying the extended depth of field video frames.

2. The method of claim 1, wherein, for a given extended depth of field video frame generated from a given set of the video frames, a pixel value of the given extended depth of field video frame is a function of corresponding pixel values of the given set of the video frames and corresponding values of focus maps generated for the given set of the video frames.

3. The method of claim 1, wherein the at least one smoothing filter comprises at least one spatial filter.

4. The method of claim 1, wherein the at least one smoothing filter comprises at least one temporal filter.

5. The method of claim 1, wherein at least one of the focus maps is generated from a luminance channel of a respective video frame.

6. The method of claim 1, wherein at least one of the focus maps is generated based on fewer than all color channels of the respective video frame.

7. The method of claim 1, wherein the extended depth of field video frames are generated in an extended depth of field mode, and the method comprises determining an excessive motion of the camera, and in response to determining the excessive motion of the camera, exiting the extended depth of field mode.

8. The method of claim 1, comprising determining an excessive motion of the camera, and in response to determining the excessive motion of the camera, reducing a number of video frames used to generate each extended depth of field video frame.

9. The method of claim 1, wherein generating the extended depth of field video frames comprises generating weights based on the filtered focus maps and combining image data from the video frames based on the weights.

10. The method of claim 9, wherein each focus map is generated based on a luminance channel of a respective video frame, and the extended depth of field video frames are generating using respective weights to combine image data from the luminance channel of the respective video frame.

11. The method of claim 1, wherein the series of video frames comprises first imaging modality video frames and second imaging modality frames, and the extended depth of field video frames are generated based only on the first imaging modality video frames.

12. The method of claim 1, comprising adjusting the focal plane setting of the camera based on at least one of the focus maps.

13. The method of claim 12, comprising determining at least one depth measurement based on the focal plane setting of the camera.

14. The method of claim 1, comprising adjusting the focal plane setting of the camera based on an output of at least one distance sensor.

15. The method of claim 1, wherein each focus map comprises an array of values corresponding to estimates of contrast in a plurality of locations of a respective video frame.

16. The method of claim 1, comprising generating estimates of tissue depth based on the focus maps.

17. The method of claim 16, wherein generating estimates of tissue depth based on the focus maps comprises determining estimates of tissue depth for different imaging modalities and comparing the estimates of tissue depth for the different imaging modalities to determine a depth of a feature of interest below a surface of tissue.

18. The method of claim 1, wherein the camera comprises a liquid lens for providing the different focal plane settings.

19. The method of claim 18, comprising controlling the liquid lens based on a camera temperature.

20. The method of claim 1, wherein the camera is an endoscopic camera.

21. The method of claim 1, wherein the camera is an open field camera.

22. The method of claim 1, comprising segmenting at least one frame and generating the extended depth of field video frames by combining image data from video frames of the series of video frames based on the segmentation of the at least one frame.

23. A system for medical imaging comprising one or more processors, at least one display, and memory storing one or more programs that include instructions for execution by the one or more processors for causing the system to:
receive a series of video frames of a region of tissue of a subject captured using different focal plane settings of a camera;
generate focus maps for at least some of the video frames of the series of video frames wherein the focus maps are generated using at least one smoothing filter;
generate extended depth of field video frames by combining image data from video frames of the series of video frames based on the focus maps; and
display the extended depth of field video frames on the at least one display.

24. A method for medical imaging comprising, at a computing system:
receiving a series of video frames of a region of tissue of a subject captured using different focal plane settings of a camera;
generating focus maps for at least some of the video frames of the series of video frames based on only luminance channels of the video frames or only one color channel of the video frames;
generating extended depth of field video frames by combining image data from video frames of the series of video frames based on the focus maps; and
displaying the extended depth of field video frames.

25. A method for medical imaging comprising:
receiving a series of video frames comprising:
first imaging modality video frames captured by a camera at one or more first imaging modality focal plane settings, and
second imaging modality video frames captured by the camera at one or more second imaging modality focal plane settings that are different from the one or more first imaging modality focal plane settings;
combining image data from at least some of the first imaging modality video frames with image data from at least some of the second imaging modality video frames to generate combined imaging modality video frames; and
displaying the combined imaging modality video frames.

26. A method for medical imaging comprising, at a computing system:
receiving a series of video frames of a region of tissue of a subject captured using different focal plane settings of a camera;
converting at least some of the video frames of the series of video frames into detail and base layers;
generating extended depth of field video frames by combining detail layers of a plurality of the video frames and combining base layers of the plurality of video frames; and
displaying the extended depth of field video frames.
